(19) United States
(12) Patent Application Publication
(10) Pub. No.: US 2026/0033996 A1
(43) Pub. Date: Feb. 5, 2026

(54) ABSORBENT ARTICLES WITH LOW TENSILE STRENGTH OUTER COVER MATERIALS

(71) Applicant: The Procter & Gamble Company, Cincinnati, OH (US)

(72) Inventors: Tanner Laurie WILLIAMS, Loveland, OH (US); Amy Lynn TALLY, Cold Spring, KY (US)

(21) Appl. No.: 19/279,151

(22) Filed: Jul. 24, 2025

Related U.S. Application Data
(60) Provisional application No. 63/677,441, filed on Jul. 31, 2024.
(60) Provisional application No. 63/702,210, filed on Oct. 2, 2024.

(51) Int. Cl. A61F 13/49

(52) U.S. Cl. CPC A61F 13/4906; A61F 2013/49098

(57) ABSTRACT

The present disclosure is directed to absorbent articles having low tensile outer cover materials and attachment of high stretch side panels to the same.

### CROSS REFERENCE TO RELATED APPLICATION

This application claims the benefit of U.S. Provisional Application No. 63/702,210, filed Oct. 2, 2024, and U.S. Provisional Application No. 63/677,441, filed Jul. 31, 2024, the substance of which are incorporated herein by reference.

### FIELD

The present disclosure is generally directed to absorbent articles and is more particularly directed to absorbent articles with low tensile strength outer cover material and side panel attachment to the same.

### BACKGROUND

It is a long-standing desire in absorbent articles for infants to include soft materials that are gentle on the baby's skin and pleasurable to the caregiver. However, there is a technical contradiction where materials, typically nonwovens, which are very soft to the touch also happen to have weak tensile strengths. Air-through bonded carded nonwovens are an example of soft nonwovens that have low tensile strengths. In some product designs this is not an issue, but in certain configurations where the outer cover material (i.e., the nonwoven that covers the liquid impermeable backsheet film) also provides attachment points for elements such as side panels, the weak tensile strengths of these materials may pose an issue. Specifically, when the absorbent article is applied, for example by a caregiver to a baby, the high stress concentrations imposed on the side panels can result in tearing of the outer cover materials and potential separation of the side panels with the remainder of the absorbent article, rendering the absorbent article unusable.

A need, therefore, exists to provide absorbent articles with high stretch side panels attached to outer covers comprising soft materials with low tensile strength.

### SUMMARY

The present disclosure solves the problems described above by using a combination of adhesives and material stack ups to reinforce the attachment of side panels to these weaker, low tensile outer cover materials and make them more resisting to tearing off. There are three concepts working together here: 1) intimately connect the outer cover material to another substrate to strengthen it as a composite, 2) introduce mechanical bonds within the vertical stack up of the side panel attachment points, and 3) selectively increase adhesives only where needed.

According to various examples, a taped absorbent article may include a front waist region, a rear waist region, a crotch region intermediate the front waist region and the rear waist region, a central longitudinal axis, a central lateral axis, a garment-facing surface, a wearer-facing surface, a liquid permeable topsheet forming a portion of the wearer-facing surface, a liquid impermeable backsheet, an absorbent core positioned at least partially intermediate the topsheet and the backsheet, the absorbent core comprising an absorbent material, an outer cover material forming a portion of the garment-facing surface, and a side panel that has a portion attached to a garment-facing side of the outer cover material in the rear waist region. The outer cover material may have a tensile strength in a direction substantially parallel to the central lateral axis in the range of about 0.5 N/cm to about 3 N/cm. The side panel may have an Elongation at 1.5N of about 25% or greater, according to the Side Panel Tensile Test. The portion of the side panel attached to a garment-facing side of the outer cover material in the rear waist region may exhibit a tearing strength when torn from the outer cover material of at least 25 N, at least 30N, or at least 35N, but less than 75N.

According to various other examples, a taped absorbent article may include a front waist region, a rear waist region, a crotch region positioned intermediate the front waist region and the rear waist region, a central longitudinal axis, a central lateral axis, a garment-facing surface, a wearer-facing surface, a liquid permeable topsheet forming a portion of the wearer-facing surface, a liquid impermeable backsheet, an absorbent core positioned at least partially intermediate the topsheet and the backsheet, the absorbent core comprising an absorbent material, an outer cover material forming a portion of the garment-facing surface, and a side panel that has a portion attached to the garment-facing side of the outer cover material in the rear waist region. A side panel adhesive may be positioned intermediate a portion of the outer cover material and the portion of the side panel. An outer cover adhesive may be positioned between the outer cover material and the backsheet. The outer cover adhesive may be distributed in an inner zone positioned proximate to the central longitudinal axis and an outer zone positioned more distal from the central longitudinal axis than the inner zone. The outer zone may at least partially overlap the side panel adhesive. A first joined area between the backsheet and the outer cover material in the outer zone may be at least 1.3 times greater than, at least 1.6 times greater than, and less than 5 times greater than, a second joined area between the backsheet and the outer cover material in the inner zone. The outer cover material may have a tensile strength in a direction substantially parallel to the central lateral axis in the range of about 0.5 N/cm to about 3 N/cm. The side panel may have an Elongation at 1.5N of about 25% or greater, according to the Side Panel Tensile Test.

According to still other examples, a taped absorbent article may include a front waist region, a rear waist region, a crotch region intermediate the front waist region and the rear waist region, a central longitudinal axis, a central lateral axis, a garment-facing surface, a wearer-facing surface, a liquid permeable topsheet forming a portion of the wearer-facing surface, a liquid impermeable backsheet, an absorbent core positioned at least partially intermediate the topsheet and the backsheet, the absorbent core comprising an absorbent material, an outer cover material forming a portion of the garment-facing surface, a side panel that has a portion attached to the garment-facing side of the outer cover material in the rear waist region, and a nonwoven strength layer positioned intermediate the outer cover and the backsheet. A side panel adhesive may be positioned intermediate a portion of the outer cover material and the portion of the side panel. An outer cover adhesive may be positioned between the outer cover material and the backsheet. The outer cover adhesive may be distributed in an inner zone positioned proximate the central longitudinal axis and an outer zone positioned more distal from the central longitudinal axis than the inner zone. The outer zone may at least partially overlap the side panel adhesive. A first joined area between the backsheet and the outer cover material in the outer zone may be greater than a second joined area between the backsheet and the outer cover material in the inner zone. The outer cover material may have a tensile strength in a direction substantially parallel to the central lateral axis in the range of about 0.5 N/cm to about 3 N/cm. The side panel may have an Elongation at 1.5N of about 25% or greater, according to the Side Panel Tensile Test.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings.

FIG. 1 is a plan view of an example absorbent article in the form of a taped diaper, garment-facing surface facing the viewer, in a flat laid-out state.

FIG. 2 is a plan view of the example absorbent article of FIG. 1, wearer-facing surface facing the viewer, in a flat laid-out state.

FIG. 3 is a front perspective view of the absorbent article of FIGS. 1 and 2 in a fastened position.

FIG. 4 is a front perspective view of an absorbent article in the form of a pant.

FIG. 5 is a rear perspective view of the absorbent article of FIG. 4.

FIG. 6 is a plan view of the absorbent article of FIG. 4, laid flat, with a garment-facing surface facing the viewer.

FIG. 7 is a cross-sectional view of the absorbent article taken about line 7-7 of FIG. 6.

FIG. 8 is a cross-sectional view of the absorbent article taken about line 8-8 of FIG. 6.

FIG. 9 is a plan view of an example absorbent core or an absorbent article.

FIG. 10 is a cross-sectional view, taken about line 10-10, of the absorbent core of FIG. 9.

FIG. 11 is a cross-sectional view, taken about line 11-11, of the absorbent core of FIG. 9.

FIG. 12 is a plan view of an example absorbent article of the present disclosure that is a sanitary napkin.

FIG. 13 is a plan view of an example absorbent article in the form of a taped diaper, garment-facing surface facing the viewer, in a flat laid-out state.

FIG. 14 is an exploded isometric view of an example side panel.

FIG. 15 is an exploded isometric view of an example absorbent article in the form of a taped diaper.

FIG. 16 is a cross-sectional view of an example absorbent article showing attachment of side panels to the remainder of an absorbent article.

FIG. 17 is a plan view of adhesive patterns between an outer cover material and a backsheet.

FIG. 18 is a schematic view of an adhesive pattern to illustrate a method of calculating a “bond area.”

FIG. 19A is a schematic diagram showing an example of a patterned apertured topsheet.

FIG. 19B is a schematic diagram showing a cross-section of the patterned apertured topsheet of FIG. 19A.

FIG. 19C is a schematic diagram showing an example of a patterned outer cover material.

FIG. 19D is an example cross-sectional illustration of an absorbent article having an outer cover material and a cushion material.

FIG. 19E illustrates a schematic view of wrinkles along a length of a laminate with the laminate in relaxed state.

FIG. 20 illustrates a partial cross-sectional view of the structure tested in Comparative Examples 1 and 2.

FIG. 21 illustrates a partial cross-sectional view of the structure tested in Inventive Example 3.

FIG. 22A illustrates a partial cross-sectional view of the structure tested in Inventive Example 4.

FIG. 22B illustrates a partial cross-sectional view of the structure tested in Inventive Example 5.

It should be understood that the various embodiments are not limited to the examples illustrated in the figures.

### DETAILED DESCRIPTION

### Introduction

As explained above, a need exists to provide absorbent articles with high stretch side panels attached to outer covers comprising soft materials with low tensile strength. It has been discovered that merely increasing adhesive amounts is not an adequate solution, because associated drawbacks. First, the placement of such adhesives is key to provide appropriate “load sharing” between the layered materials that make up the absorbent article. Second, large quantities of adhesive may also compromise the softness of the outer cover material, negating the original intent of the material choice.

Various non-limiting forms of the present disclosure will now be described to provide an overall understanding of the principles of the structure, function, manufacture, and use of various types of absorbent articles generally and, more specifically, absorbent articles with low tensile strength outer cover materials. One or more examples of these non-limiting forms are illustrated in the accompanying drawings. Those of ordinary skill in the art will understand that the absorbent articles with low tensile strength outer cover materials described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### Definitions

Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Disposed on” refers to a positional state indicating that one object or material is arranged in a position adjacent to the position of another object or material. The term does not require or exclude the presence of intervening objects, materials, or layers.

“Absorbent article” refers to devices that absorb and contain liquid, and more specifically, refers to devices that are placed against or in proximity to the body of the wearer to absorb and to contain various exudates discharged from the body.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

“High-stretch” refers to an elastically extensible substrate having an elongation of 25% or greater at 1.5 N as measured by the Side Panel Tensile Test described herein.

“Low-tensile” refers to a material having a tensile strength in the range of about 0.5 N/cm to about 3 N/cm as measured by the Outer Cover Tensile Test described herein.

### General Description of an Absorbent Article

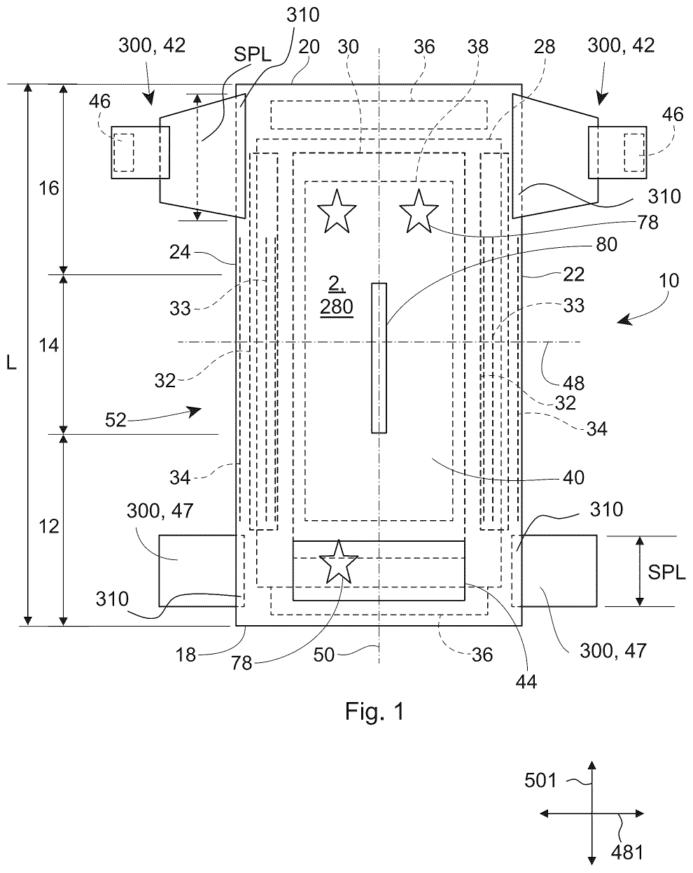

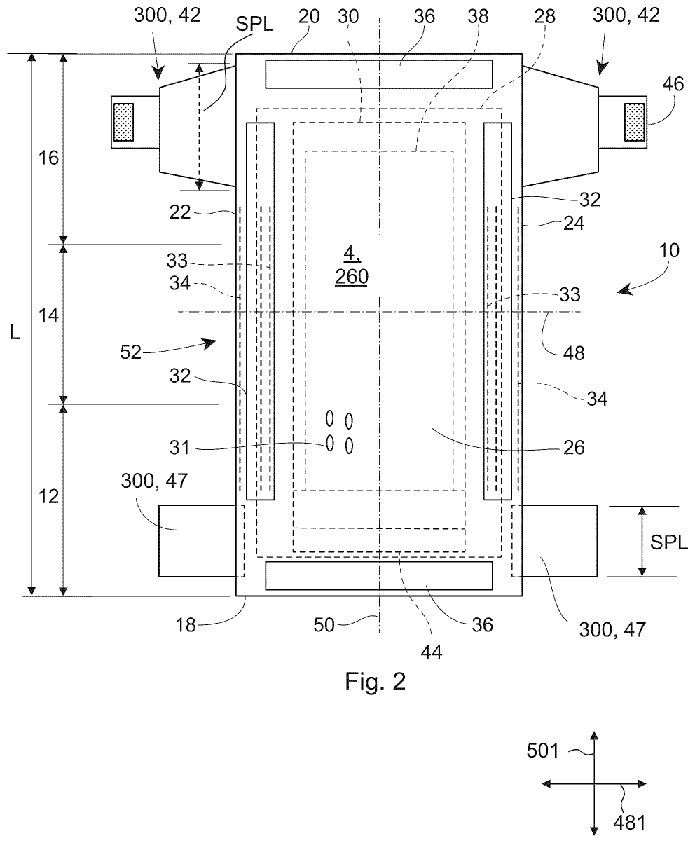

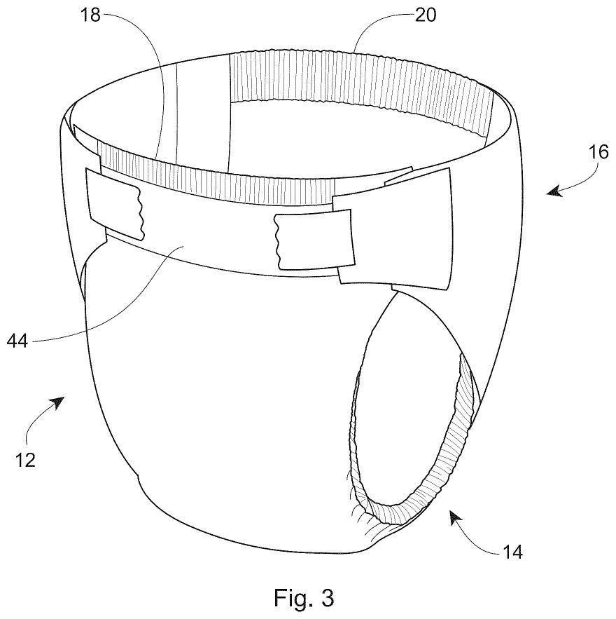

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28.

The absorbent article 10 may comprise one or more side panels 300, such as, for example back ears 42 and/or front ears 47. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. A first direction 501 may be defined that extends substantially along or substantially parallel to the central longitudinal axis 50. The absorbent article 10 may comprise a total longitudinal length, L, measured in the first direction 501, substantially along or parallel to the central longitudinal axis 50. Similarly, each of the one or more side panels 300 may comprise a total longitudinal length, SPL, measured in the first direction 501, substantially along or parallel to the central longitudinal axis 50. The central lateral axis 48 extends perpendicular to the central longitudinal axis 50. A second direction 481 may be defined that extends substantially along or substantially parallel to the central lateral axis 48. Each of the one or more side panels 300 may be extensible in at least the second direction 481, substantially along or parallel to the central lateral axis 48.

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137.

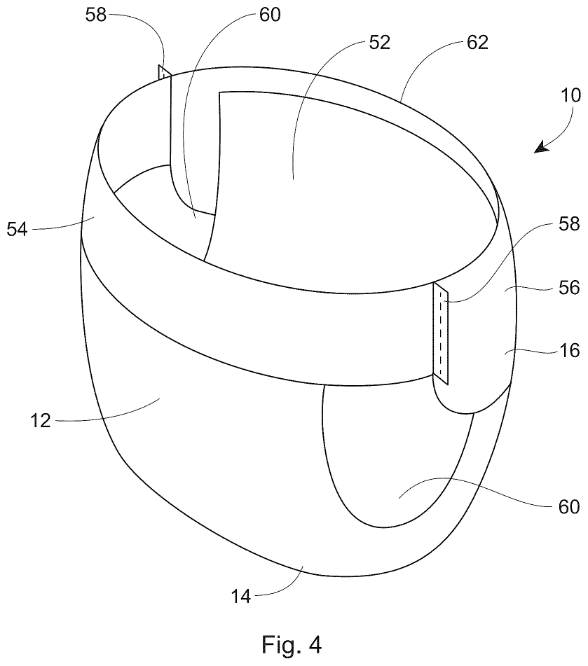

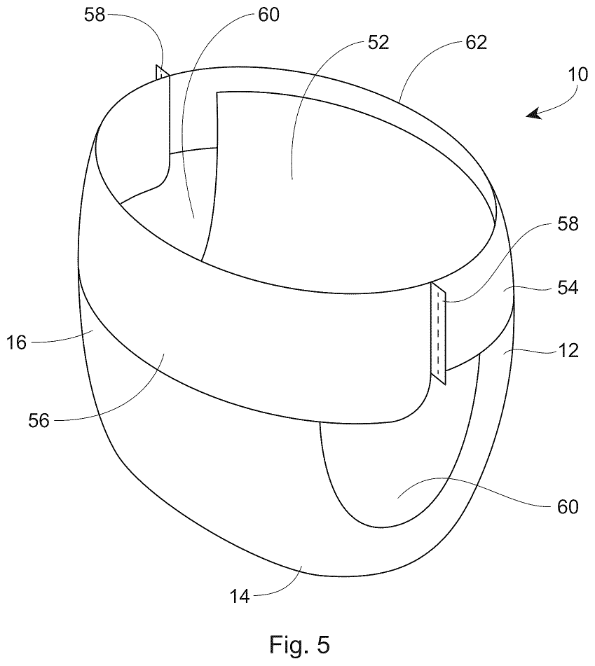

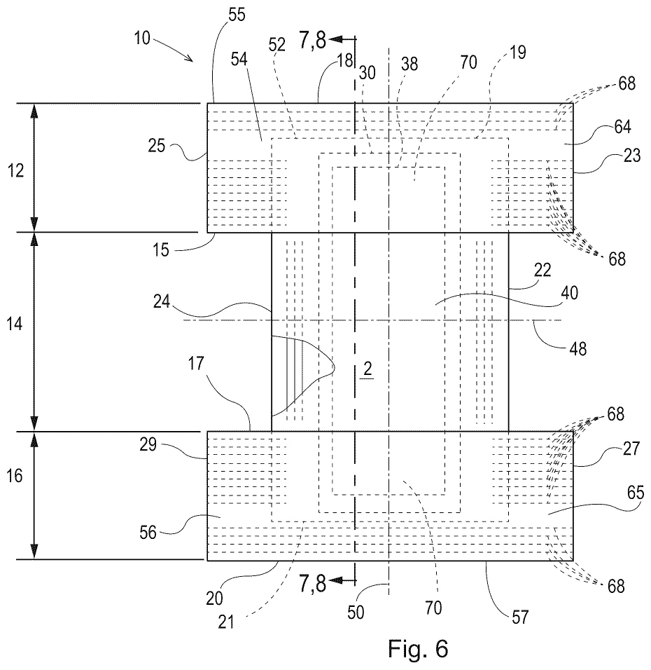

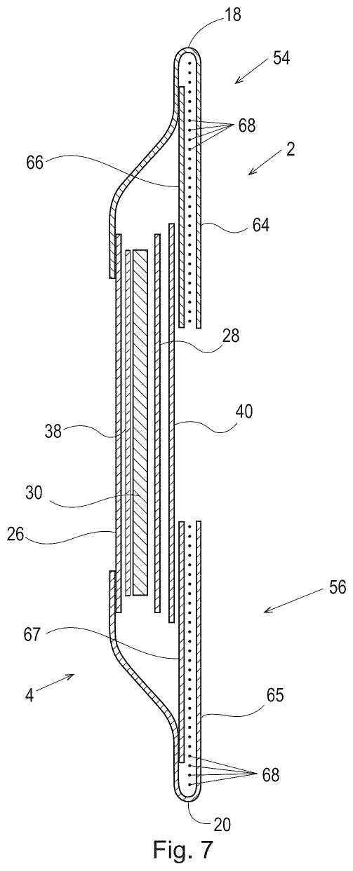

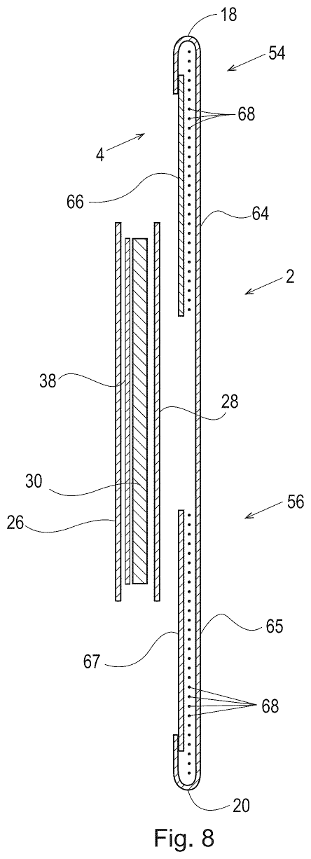

Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. No. 9,072,632.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8. In some configurations shown in FIGS. 7 and 8, portions of the outer belt layers 64, 65 may be folded over onto the inner belt layers, respectively. In addition, as shown in FIG. 7, portions of the outer belt layers 64, 65 may also be folded over onto the chassis 52.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt edges 15 and 17; or, alternatively, adjacent to the seams 58 and/or proximal front and back belt edges 15 and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may comprise a variable basis weight nonwoven material, such as those described in U.S. Pat. Appl. No. 2017/0191198. The topsheet may be apertured by overbonding a material and then rupturing the overbonds through ring rolling, such as disclosed in U.S. Pat. No. 5,628,097, to Benson et al., issued on May 13, 1997, and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The topsheet may comprise a one or more layer hydroentangled material with or without apertures. The topsheet may comprise a variable basis weight nonwoven material. Referring to FIG. 2, at least a portion of the wearer-facing surface 4 may comprise a liquid permeable portion 260 formed by the topsheet 26.

The topsheet may be a patterned apertured topsheet. Example topsheets with patterns of apertures are disclosed in U.S. Pat. No. 11,090,202, issued on Aug. 17, 2021, U.S. Pat. No. D851414, issued on Jun. 18, 2019, and U.S. Pat. No. D895303, issued on Sep. 8, 2020.

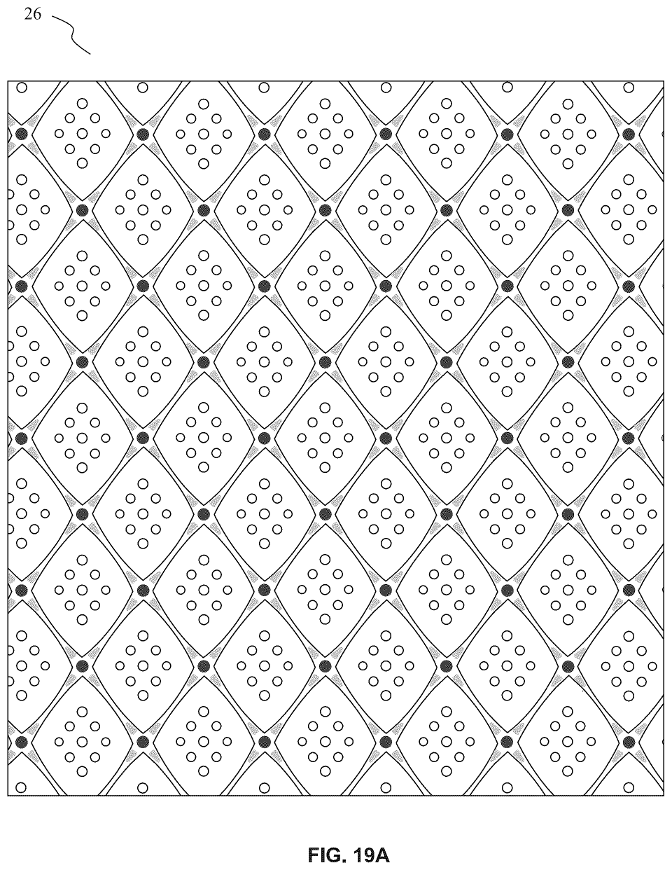

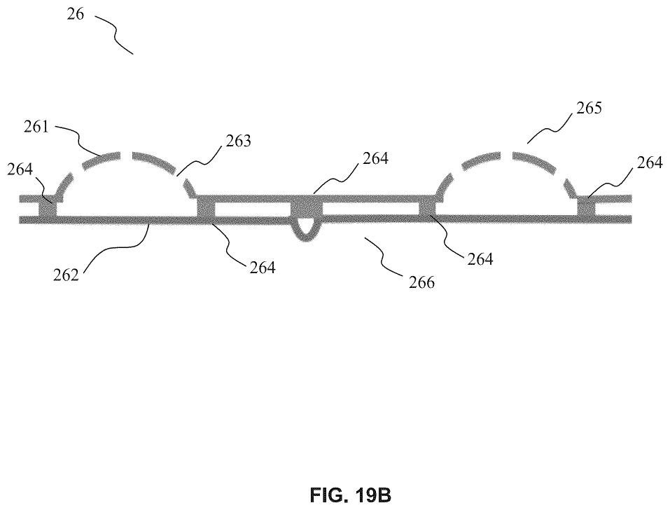

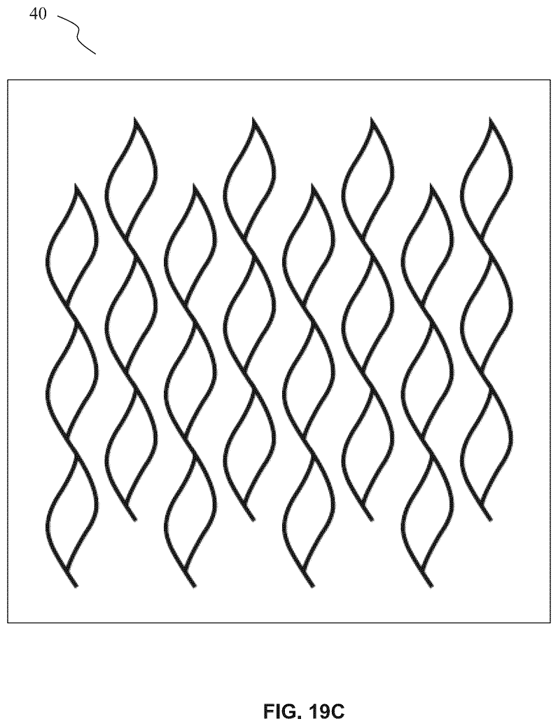

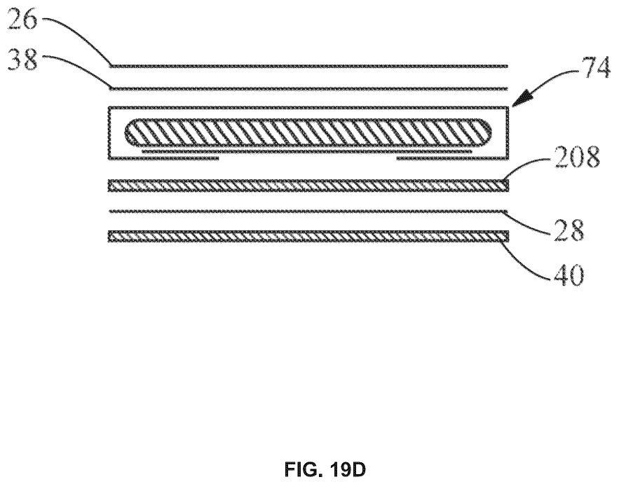

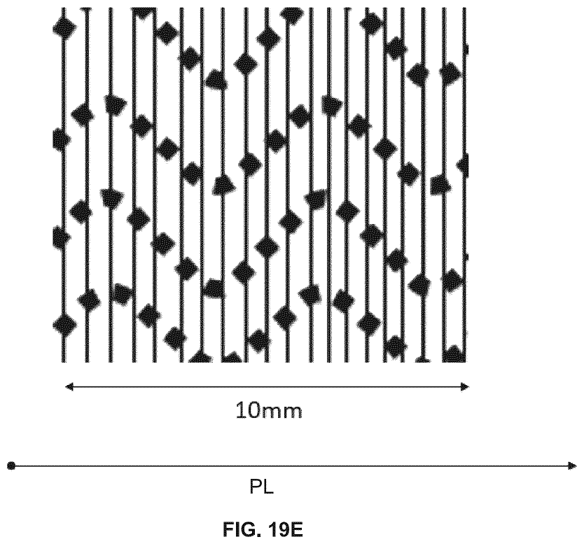

FIG. 19A is a schematic diagram showing an example of a patterned apertured topsheet.

FIG. 19B is a schematic diagram showing a cross-section of the patterned apertured topsheet of FIG. 19A. As can be seen from FIG. 19B, the topsheet 26 may comprise multiple layers, including for example a top layer 261 and bottom layer 262. The top layer 261 may be a 100% hydrophilic 2.6 denier eccentric PE/PET bicomponent fiber and may have a basis weight of 20 gsm, determined by the Basis Weight Test described hereinafter. The bottom layer 262 may be a carded nonwoven comprising 60% of 2 denier concentric hydrophilic PE/PET bicomponent fibers having a basis weight of 20 gsm and 40% of 4 denier concentric hydrophilic PE/PET bicomponent fibers having a basis weight of 42 gsm, the basis weights being determined by the Basis Weight Test described hereinafter. The bottom layer 262 may be deformed and need not be flat before the top layer is bonded to the bottom layer bonded. The topsheet 26 may include raised portions 265 and compressed regions 266. Apertures 263 in may be present in the raised portions 265 and ultrasonic bonds 264 may be present in the compressed regions 266. Other embodiments may not include ultrasonic bonds 264 in the compressed regions 266.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 28 may be coterminous with the outer cover material 40.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. Referring to FIG. 1, and depending on the composition of the outer cover material 40, at least a portion of the garment-facing surface 2 may comprise a low low-tensile portion 280 formed by at least a portion of the outer cover material 40.

### Outer Cover Material Composition

The outer cover material 40 may be a hydroentangled nonwoven material or a variable basis weight nonwoven material. Such a material may have one or more layers. The outer cover material 40 may be a carded nonwoven material. The outer cover material 40 may comprise a bond pattern, apertures, and/or three-dimensional features.

### Outer Cover Material Softness

Softness, texture (i.e., smoothness), and/or stiffness of a garment-facing surface of an outer cover material 40 may be measured by an Emtec Tissue Softness Analyzer, according to the Emtec Test herein. Tactile softness is measured as TS7. Texture/Smoothness is measured as TS750. Stiffness is measured as D. A portion of, or all of, garment-facing sides or surfaces 43 of the outer cover nonwoven materials 40 of the present disclosure may have a TS7 value in the range of about 1 dB V² rms to about 8 dB V² rms, about 1 dB V² rms to about 6 dB V² rms, about 2 dB V² rms to about 4.5 dB V² rms, or about 2 dB V² rms to about 4.0 dB V² rms. The portion of, or all of, the garment-facing surfaces of the outer cover materials of the present disclosure may also have a TS750 value in the range of about 2 dB V² rms to about 30 dB V² rms, about 6 dB V² rms to about 30 dB V² rms, about 6 dB V² rms to about 20 dB V² rms, about 6 dB V² rms to about 15 dB V² rms, about 6 dB V² rms to about 12 dB V² rms, or about 6.5 dB V² rms to about 10 dB V² rms. The portion of, or all of, the garment-facing surfaces of the outer cover materials of the present disclosure may also have a D value in the range of about 1 mm/N to about 10 mm/N, about 3 mm/N to about 8 mm/N, about 2 mm/N to about 6 mm/N, about 2 mm/N to about 4 mm/N, or about 3 mm/N to about 4 mm/N. All values are measured according to the Emtec Test herein.

### Outer Cover Material Tensile Strength

The outer cover material 40 may have a tensile strength in a direction 481 substantially parallel to the central lateral axis 48 in the range of about 0.5 N/cm to about 3 N/cm, or about 0.6 N/cm to about 2.9 N/cm, or about 0.7 N/cm to about 2.8 N/cm, or about 0.8 N/cm to about 2.7 N/cm, or about 0.9 N/cm to about 2.6 N/cm, or about 1. N/cm to about 2.5 N/cm, or about 1.1 N/cm to about 2.4 N/cm, or about 1.2 N/cm to about 2.3 N/cm, or about 1.3 N/cm to about 2.2 N/cm, or about 1.4 N/cm to about 2.1 N/cm, or about 1.5 N/cm to about 2 N/cm, or about 1.6 N/cm to about 1.9 N/cm, or about 1.7 N/cm to about 1.8 N/cm. All values are measured according to the Outer Cover Tensile Test herein. This direction 481 may substantially correspond with a direction in which the side panels 300 that are attached to the outer cover material 40 are stretched when the absorbent article 10 is applied to a wearer.

### Outer Cover Material Denier

A denier of fibers of the outer cover material 40 may be in the range of about 0.4 denier to about 3 denier, or about 0.5 denier to about 2.9 denier, or about 0.6 denier to about 2.8 denier, or about 0.7 denier to about 2.7 denier, or about 0.8 denier to about 2.6 denier, or about 0.9 denier to about 2.5 denier, or about 1. denier to about 2.4 denier, or about 1.1 denier to about 2.3 denier, or about 1.2 denier to about 2.2 denier, or about 1.3 denier to about 2.1 denier, or about 1.4 denier to about 2 denier, or about 1.5 denier to about 1.9 denier, or about 1.6 denier to about 1.8 denier, or about 1.7 denier. Denier is a standard unit of measurement used to describe the thickness fibers.

### Outer Cover Material Basis Weight

A basis weight of the outer cover material 40 may be in the range of about 15 gsm to about 40 gsm, or about 16 gsm to about 39 gsm, or about 17 gsm to about 38 gsm, or about 18 gsm to about 37 gsm, or about 19 gsm to about 36 gsm, or about 20 gsm to about 35 gsm, or about 21 gsm to about 34 gsm, or about 22 gsm to about 33 gsm, or about 23 gsm to about 32 gsm, or about 24 gsm to about 31 gsm, or about 25 gsm to about 30 gsm, or about 26 gsm to about 29 gsm, or about 27 gsm to about 28 gsm. All values are measured according to the Basis Weight Test Method.

### Outer Cover Material Variations

The outer cover material may comprise a pattern or may have a flat, non-patterned texture. FIG. 19C is a schematic diagram showing an example of a patterned outer cover material 40. More specifically, FIG. 19C shows an outer cover material made from a carded nonwoven having an embossed pattern. The embossed pattern includes a plurality of wavy elements.

An example of a specific outer cover material that may include the pattern shown in FIG. 19C, may be a carded nonwoven comprising combination of 50% 0.8 denier PE/PET bicomponent fibers and 50% 1.2 denier PE/PET bicomponent fibers. The nonwoven may be air through cured with heat and pressure embossing, which may create the wavy pattern. An anti-static treatment may be applied to the material.

An example of a specific outer cover material that may have a flat pattern, may be a carded nonwoven comprising 50% 0.8 denier PE/PET bicomponent fibers and 50% 1.2 denier PE/PET bicomponent fibers. The fibers in the flat material may be layered in the structure. This outer cover material may be air through carded. An anti-static treatment may be applied to the material.

### Absorbent Core

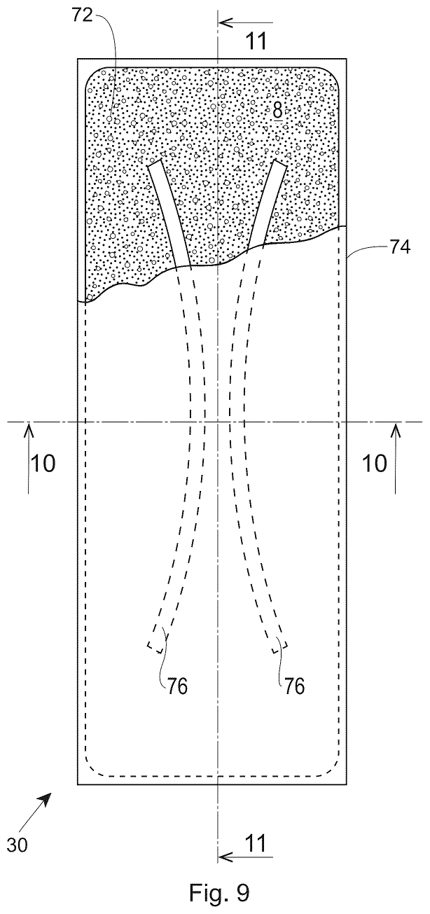

As used herein, the term “absorbent core” refers to a component of the absorbent article 10 for absorbing and containing liquid such as urine received by the absorbent article. The absorbent core thus typically has a high absorbent capacity. An example absorbent core 30 is schematically shown in FIGS. 9-11. The absorbent core comprises an absorbent material 72. The absorbent typically comprises a core wrap 74 that encloses or sandwiches the absorbent material.

The core wrap may be a single material that is folded and attached to itself, or it may comprise a separate top layer and bottom layer that may be bonded, adhesively joined, or otherwise joined together. The top and bottom layers of the core wrap may be the same or different. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam. High loft nonwoven materials may be present in the core bags, or proximate but outside the core bags. The absorbent material may comprise one or more layers of a coform material. The coform material may comprise a mixture of fibers and an absorbent material. The fibers may comprise staple fibers such as synthetic fibers or absorbent fibers. The synthetic fibers may comprise polypropylene fibers, polyethylene fibers, and/or bicomponent fibers. The absorbent fibers may comprise pulp, lyocell, and/or viscose. The fibers may also comprise thermoplastic filaments (scattered or interconnected networks). The absorbent material may comprise a super absorbent polymeric material in fiber or particle form. The one or more layers of coform material may comprise virgin or recycled materials. The staple fibers may be present in the coform material in an amount of from about 5 wt. % to about 50 wt. %. The absorbent material may be present in the nonwoven web in an amount of from about 50 wt. % to about 95 wt. %. The staple fibers may have an average length of from about 5 mm to about 50 mm. The staple fibers and absorbent fibers may be thermally bonded or hydraulically entangled to form the nonwoven web (as disclosed in WO2023/022979).

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from about 20 g/g to about 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. No. 7,838,722 (Blessing), U.S. Pat. Nos. 9,072,634 and 8,206,533 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate, such as a carded nonwoven layer, having a porous structure into which SAP particles have been deposited, may also be used in present disclosure.

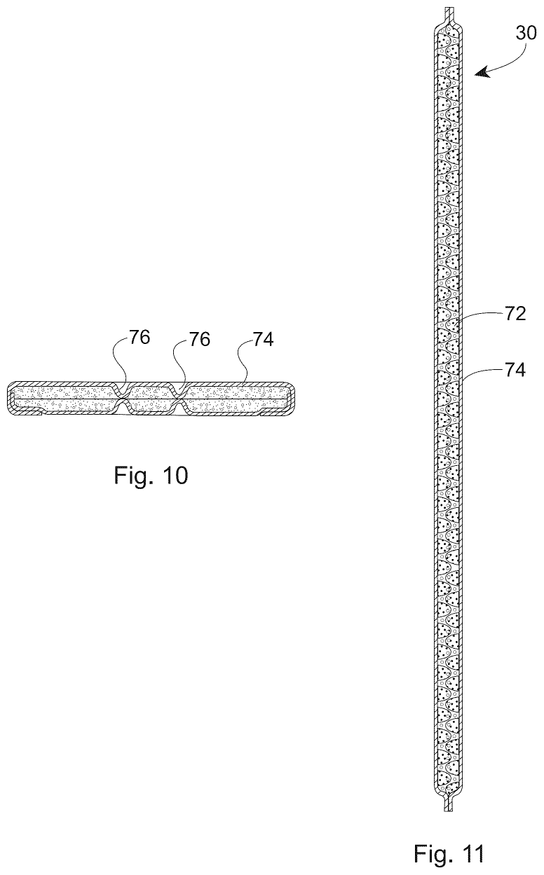

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/0312622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in U.S. Pat. No. 9,789,011. One or more channels may also be formed in absorbent cores comprising a mix of cellulose fibers and SAP particles. These channels may embody any suitable shapes and any suitable number of channels may be provided. In other instances, the absorbent core may be embossed to create the impression of channels, with absorbent material remaining within the channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14. The barrier leg cuffs 32 may comprise one or more layers of nonwoven material. The nonwoven material may be hydroentangled.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. Alternatively, the elastic waistbands may be positioned intermediate the topsheet and the backsheet. As an example, a first elastic waistband 36 may be present in the front waist region 12 near the front end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The elastic waistband may comprise an elastic film joined to the topsheet and a nonwoven material covering the elastic film. In other instances, the elastic waistband may comprise an elastic film sandwiched between two nonwoven materials. The elastic film may be ultrasonically bonded, or otherwise bonded or attached, to the one or more nonwoven materials. The one or more nonwoven materials may be hydroentangled. The elastic film and/or the nonwoven materials may be preactivated (i.e., activated prior to being joined together) or the formed elastic film/nonwoven laminate may be activated post laminate formation.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. The acquisition material may be rectangular or may be shaped, such as hourglass shaped. Typically, an acquisition material 38 may have a width and length that are smaller than the width and length of the topsheet 26. The acquisition material may be a secondary topsheet in the feminine pad context. The acquisition materials may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Side Panels: Front Ears and Back Ears

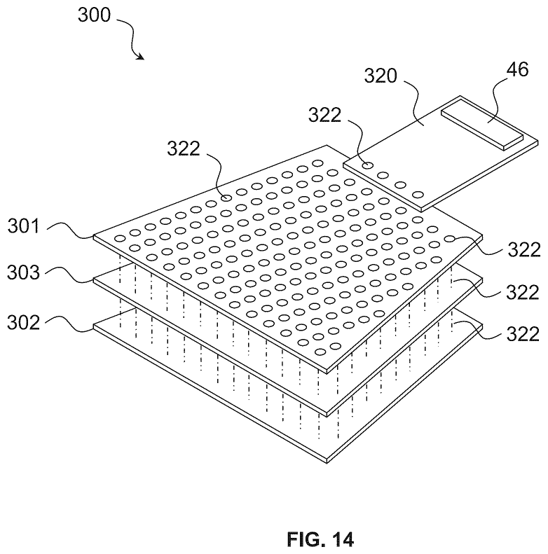

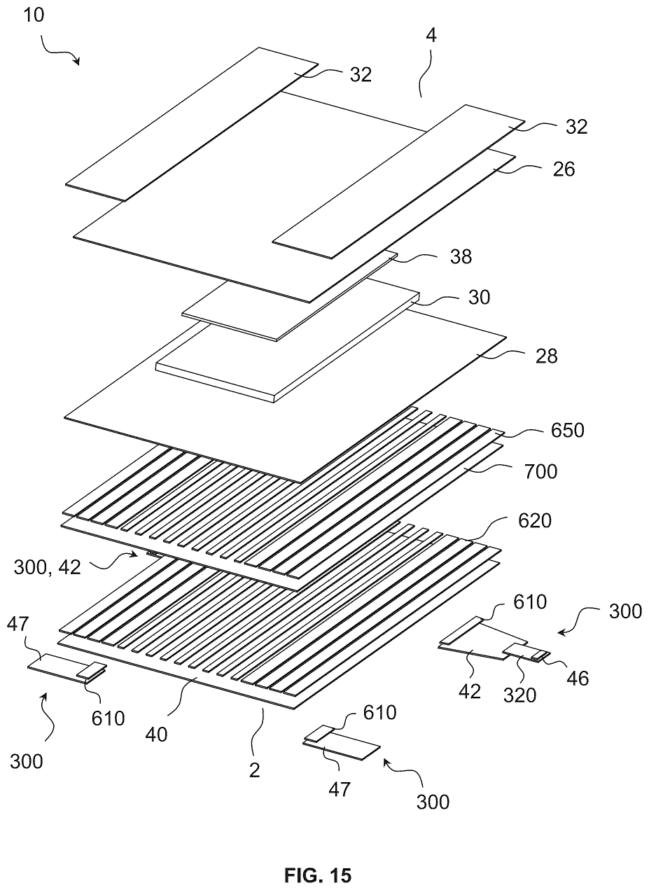

Referring to FIGS. 1, 2, and 14, as referenced above, the absorbent article 10 may have one or more side panels 300, which may have any structure or function, but which may be, for example, front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. Any ears with fasteners may be configured to engage an opposing ear or the landing zone or landing zone area 44. One set of ears may comprise primary fasteners and the other set of ears may comprise secondary fasteners. The ears, or portions thereof, may be elastic or may have elastic panels. In an example, an elastic film 303 or elastic strands may be positioned intermediate a first nonwoven 301 material and a second nonwoven material 302. One or more of the first and second nonwoven materials may be hydroentangled. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. The back ears may comprise an elastic film sandwiched between two nonwoven materials forming a laminate. The elastic film and/or the nonwoven materials may be pre-activated (i.e., activated prior to being joined together) and the laminate may be joined by ultrasonic bonds. Such laminates are disclosed in U.S. Pat. No. 10,485,713. In other instances, the elastic film may not be preactivated and instead the laminate may be activated after the laminate is formed. Such laminates may also be ultrasonically bonded via one or more ultrasonic bonds 322, as shown in FIG. 14. According to some examples, the fasteners 46 may be positioned on a tape tab 320, which may, in turn, be bonded to the side panel 300 by any of the bonding techniques mentioned above, including but not limited to ultrasonic bonding. Referring to FIG. 1, each of the one or more side panels 300 may be attached to the absorbent article 10. For example, the back ears 42 may be attached to the outer cover material 40 at one or more attachment portions 310.

### Side Panel to Absorbent Article Length Ratio

As previously described with respect to FIG. 1, the taped absorbent article 10 may have a total longitudinal length, L, taken in a direction 501 parallel to the central longitudinal axis 50 and the side panel 300 may have a total longitudinal length, SPL, at its greatest length, taken in a direction 501 parallel to the central longitudinal axis 50. According to various examples, the SPL may be about 15% to about 30%, or about 16% to about 29%, or about 17% to about 28%, or about 18% to about 27%, or about 19% to about 26%, or about 20% to about 25%, or about 21% to about 24%, or about 22% to about 23% of L.

### Side Panel Elongation

The side panel 300 may have an Elongation at 1.5N of about 25% or greater, according to the Side Panel Tensile Test. For example, the side panel 300 may have an Elongation at 1.5N of about 25% to about 100%, or about 30% to about 95%, or about 35% to about 90%, or about 40% to about 85%, or about 45% to about 80%, or about 50% to about 75%, or about 55% to about 70%, or about 60% to about 65%.

### Side Panel Unload Force

The taped absorbent article 10 of any one of the preceding claims, wherein the side panel 300 may have an Unload Force at 50% of at least about 0.9 N to about 1.5 N, or about 1 to about 1,4 N, or about 1.1 to about 1.3 N, or about 1.2, according to the Hysteresis Test.

### Side Panel Hysteresis Ratio

The taped absorbent article 10 of any one of the preceding claims, wherein the side panel 300 may have a Hysteresis Ratio of about 2 to about 4, or about 2.1 to about 3.9, or about 2.2 to about 3.8, or about 2.3 to about 3.7, or about 2.4 to about 3.6, or about 2.5 to about 3.5, or about 2.6 to about 3.4, or about 2.7 to about 3.3, or about 2.8 to about 3.2, or about 2.9 to about 3.1, or about 3 about 3, according to the Hysteresis Test.

### Side Panel Tearing Strength

The side panel 300 may have an attachment portion 310 attached to a garment-facing side 41 of the outer cover material 40 in the back waist region 16. A tearing strength of the attachment portion 310 of the side panel 300 from the outer cover material 40 may be at least 25 N, at least 30N, or at least 35N, but less than 75N. For example, the tearing strength of the attachment portion 310 of the side panel 300 from the outer cover material 40 may be about 25 N to about 75 N, or about 30 to about 70 N, or about 35 to about 65 N, or about 40 to about 60 N, or about 45 to about 55 N, or about 50.

### Side Panel Wrinkle Distribution

As expressed above, elastically elongatable panels 300 may be joined to the chassis 52, for example as described above. The side panels 300 may comprise: at least one, or at least two, cover layers 301, 302 and an elastomeric film 303 attached to the cover layer 301, wherein said least one cover layer 301 and elastomeric film 303 are joined by mechanical bonding, at a plurality of discrete bonding elements 322, preferably having a shape selected from substantially square, rectangular, circular and combinations thereof, and wherein each said panel comprises a plurality of wrinkles having peaks and troughs formed on at least one, preferably both, of said cover layer, wherein said panel comprises an average of no more than two, preferably no more than one, wrinkles between two consecutive discrete bonding elements along a panel length extending substantially parallel to the transverse axis, and wherein said panel comprises a wrinkle distribution of at least 0.5, from 0.7 to 11, even from 0.8 to 1, wrinkles/mm, according to the method herein. Advantageously, a large number of wrinkles within the above ranges generally increases bulkiness and therefore actual and perceived softness of the material which is ever more important in elastic side panels which are in tight contact with the skin of the user, nevertheless if the number of wrinkles is too high bulkiness turns into roughness as the peaks tend to form sharper apexes at extremely short amplitudes therefore it is beneficial to even more preferably remain within the above cited preferred range.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Packages

The absorbent articles of the present disclosure may be placed into packages. The packages may comprise polymeric films, paper, and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages. The packages may comprise polymeric films comprising recycled material, such as about 20% to about 100%, about 30% to about 90%, about 30% to about 80%, about 40% to about 60%, or about 50% recycled material. The recycled material may comprise post-industrial recycled material (PIR) and/or post-consumer recycled material (PCR). In some instances, the polymeric films used for the packages may comprise two outer layers and one or more inner layers. The one or more inner layers may comprise the recycled material or may comprise more recycled material than the outer layers. The recycled material may comprise recycled polyethylene. The recycled material may comprise recycled polyethylene PIR from trim from the packaging operation.

The package material may comprise paper, paper based material, paper with one or more barrier layers, or a paper/film laminate. The package material may be in the range of about 50 gsm to about 100 gsm or about 70 gsm to about 90 gsm and the one or more barrier layers may be in the range of about 3 gsm to about 15 gsm. The paper based package material with or without one or more barrier layers may exhibit a machine direction tensile strength of at least 5.0 kN/m, a machine direction stretch of at least 3 percent, a cross-machine direction tensile strength of at least 3 kN/m, and a cross-direction stretch at break of at least 4 percent, each as determined via ISO 1924-3.

The paper based package material or paper based package material comprising a barrier layer or film may be recyclable or recyclable in normal paper recycling operations. The recyclability extent of the paper based package may be determined via recyclable percentage. The paper based package of the present disclosure may exhibit recyclable percentages of 70 percent or greater, 80 percent or greater, or 90 percent or greater. The paper based package of the present disclosure may have a recyclable percentage of between 70 percent to about 99.9 percent, between about 80 percent to about 99.9 percent, or between about 90 percent to about 99.9 percent. In one example, the package material of the present disclosure may exhibit a recyclable percentage of from about 95 percent to about 99.9 percent, from about 97 percent to about 99.9 percent, or from about 98 percent to about 99.9 percent. The recyclable percentage of the paper based package may be determined via test PTS-RH:021/97 (Draft Oct. 2019) under category II, as performed by Papiertechnische Stiftung located at Pirnaer Strasse 37, 01809 Heidenau, Germany. In another instance, the paper based packages of the present disclosure may exhibit an overall “pass” test outcome as determined by PTS-RH:021/97 (Draft Oct. 2019) under category II method. Any of the paper based packages may have opening features, such as lines of perforation, and may also have handles.

### Arrays

“Array” means a display of packages comprising disposable absorbent articles of different article constructions (e.g., different elastomeric materials [compositionally and/or structurally] in the side panels, back ears, side flaps and/or belts flaps, different graphic elements, different product structures, fasteners, waistbands, or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Sanitary Napkin

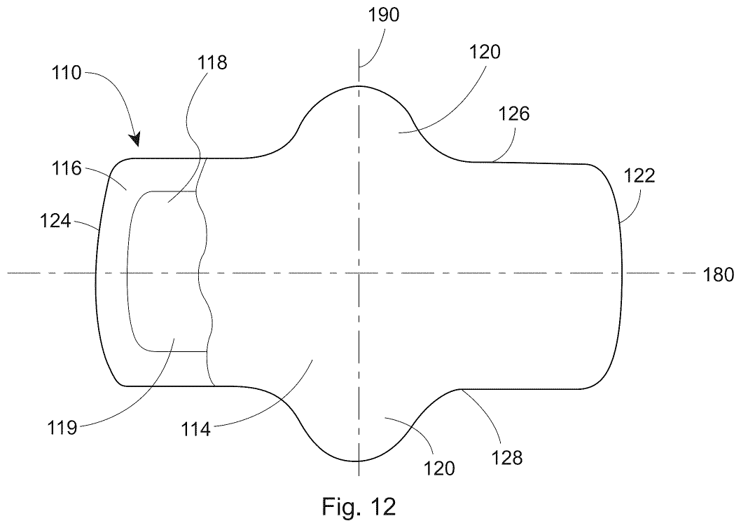

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Fasteners

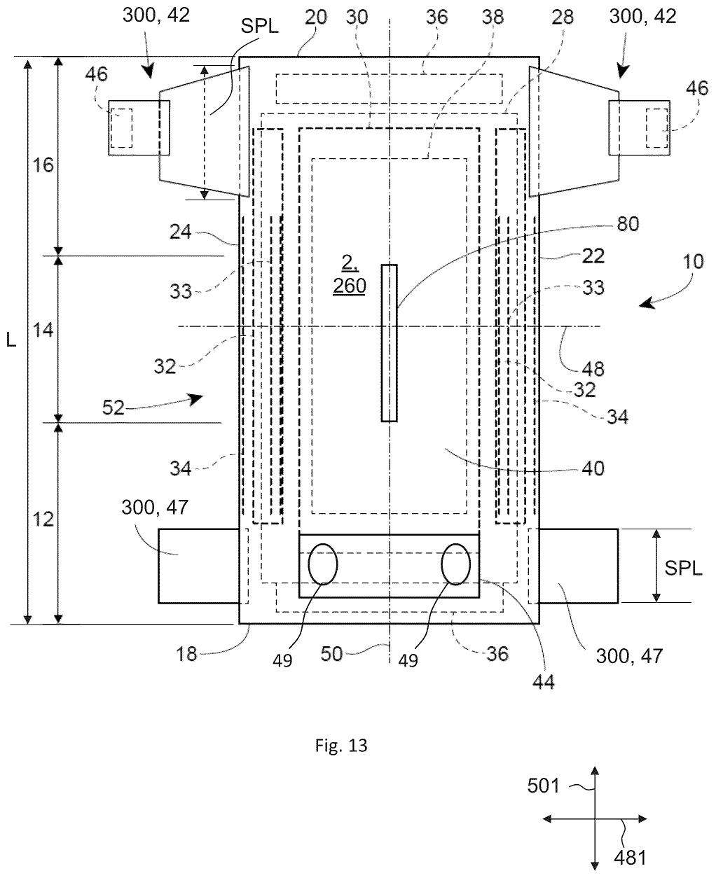

Referring to FIG. 13, the back ears 42 may comprise fasteners 46. The fasteners 46 may be configured to cooperate with the landing zone area or landing zone material 44 on the garment-facing surface of the front waist region 12 of the absorbent article 10. Additionally, the absorbent article may comprise one or more secondary fasteners 49. The secondary fasteners 49 may be disposed on the outer cover material 40 or a component of the absorbent article, such as the landing zone 44 as illustrated in FIG. 13. The secondary fasteners may be a separate material joined to the absorbent article. The secondary fasteners may be integrally formed from a material of the absorbent article. For example, the secondary fasteners may comprise one or more hooks or protrusions formed from the material of the outer cover material 40, the landing zone material 44, or a film. The hooks or protrusions may be formed using the process described in U.S. Pat. No. 8,784,722 to Rocha et al. The secondary fasteners may be configured to cooperate with a portion of the back ears. For example, a secondary fastener comprising hooks or protrusions are configured to engage the nonwoven material of the back ear. It is to be appreciated that the secondary fasteners may be any mechanical fastener that is configured to cooperate with a component of the absorbent article.

### High-Stretch Side Panels Bonded to Soft, Low-Tensile Outer Cover Materials

As described above, a need exists to provide absorbent articles 10 with high-stretch side panels 300 attached to outer covers 40 comprising soft materials with low tensile strength. One could imagine a solution where increasing adhesive amounts is the sole solution to improve this attachment, but it has been discovered that this solution may introduce unacceptable tradeoffs. First, the placement of such adhesives is key to provide appropriate “load sharing” between materials. Second, large quantities of adhesive may compromise the softness of the outer cover material 40, negating the original intent of the material choice.

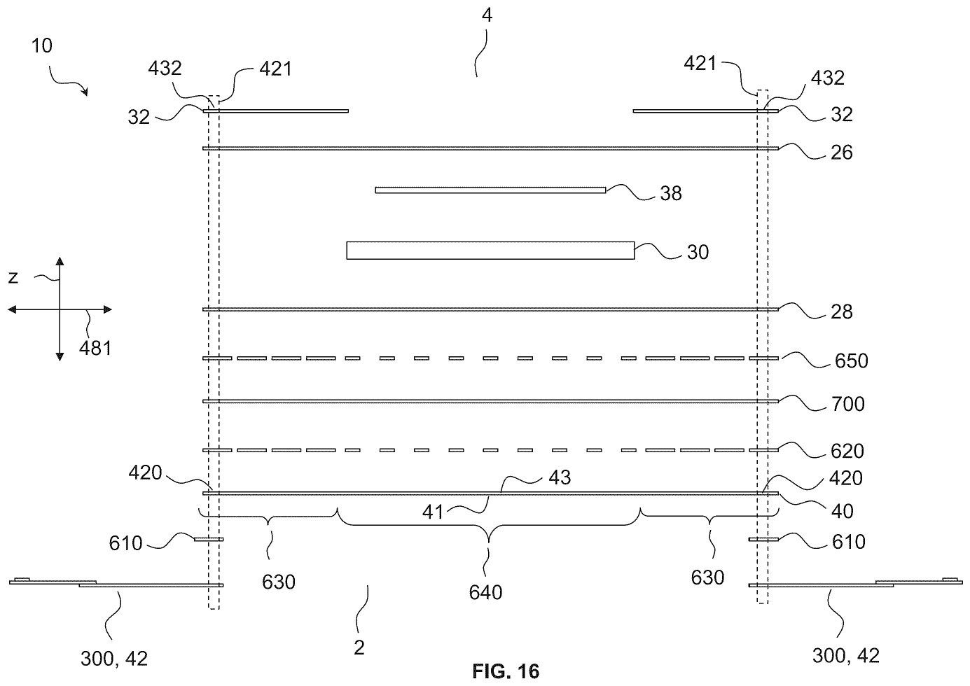

FIG. 15 is an exploded isometric view of an example absorbent article 10 in the form of a taped diaper. FIG. 15 focuses on the positions and interrelationships between some layers without intending to imply that layers or structures not shown must be omitted. FIG. 16 is a cross-sectional view of an example absorbent article 10 showing attachment of side panels 300 to the remainder of an absorbent article 10. The absorbent article 10 may comprise barrier leg cuffs 32, a top sheet 26, acquisition materials 38, an absorbent core 30, a backsheet 28, and an outer cover material 40. Side panels 300, such as for example, one or more back ears 42 and one or more front ears 47, may also be provided. The back ears 42 may comprise a tape tab 320 that includes fasteners 46. One or more of the side panels 300 may be affixed to the outer cover material 40 at a garment facing surface 2 of the absorbent article 10 by a side panel adhesive 610. The outer cover material 40 may be affixed to the absorbent article 10 by an outer cover adhesive 620. The outer cover adhesive 620 may bond the outer cover material 40 directly to the backsheet 28 or to an intervening nonwoven strength layer 700, which may be positioned between the outer cover material 40 and the backsheet 28. The nonwoven strength layer 700, if present, maybe bonded to the backsheet 28 by a strength layer adhesive 650.

Referring to the cross-section shown in FIG. 16, a plurality of the layers of the absorbent article 10 may be bonded together via one or more mechanical bonds 421. For example, the barrier leg cuffs 32 may comprise a bonded-overlapped portion 432 and the outer cover material 40 may comprise a mechanically-bonded portion 420. As shown, the mechanical bonds 421 may bond other layers as well, including but not limited to topsheet 26, bottom sheet 28, strength layer 700, and side panels 300. Thus, each of these layers may comprise a mechanically-bonded portion. It is also noted with respect to the cross-section shown in FIG. 16 that just as the overall absorbent article 10 may have a garment-facing surface 2 and a wearer-facing surface 4 each layer may have a garment-facing side comprising a garment-facing surface and a wearer-facing side comprising a wearer-facing surface. For example, the outer cover material 40 may comprise a garment-facing side 41 and a wearer-facing sider 43. The outer cover adhesive 620 may be disposed on or adhered to the wearer-facing side 43 and a side panel adhesive 610 may join the side panels 300 to the garment-facing side 41 of the outer cover material 40. Similarly, the strength layer adhesive 650 may be disposed on or adhered to a wearer-facing side of the nonwoven strength layer 700 to join the nonwoven strength layer 700 to a garment-facing side of the backsheet 28.

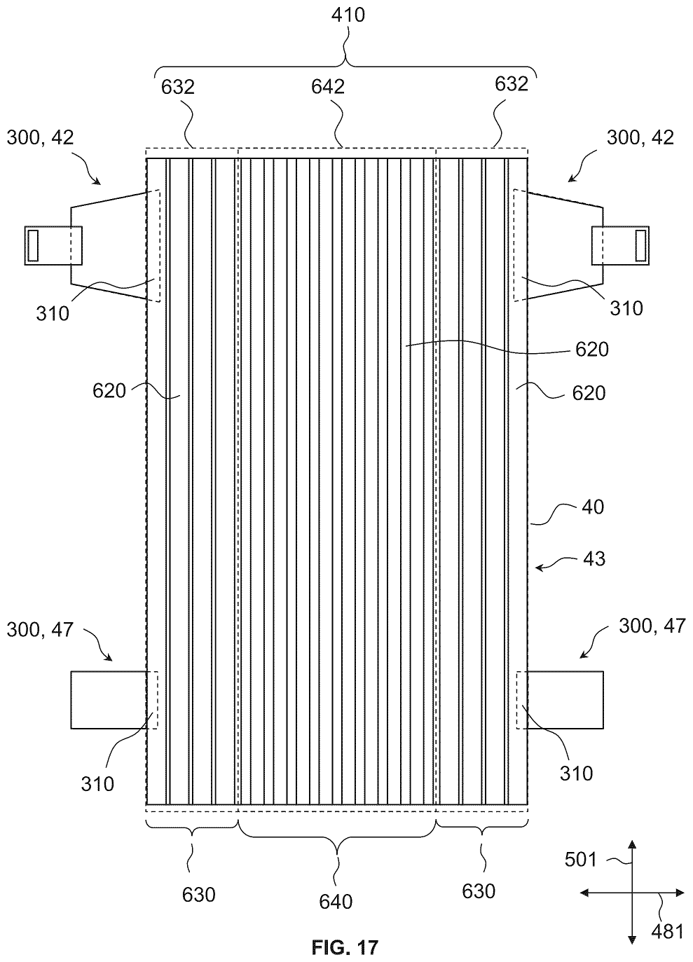

Still referring to FIG. 16 and also to FIG. 17, which is a plan view of adhesive patterns between an outer cover material 40 and a backsheet 28, the outer cover adhesive 620 and/or the strength layer adhesive 650 may be comprise a number of distinct zones 630, 640 and areas 632, 642. The zones 630, 640 may include one or more outer zones 630 and at least one inner zone 640.

These zones 630, 640 may extend in a direction 481 that is substantially parallel to the central lateral axis 48 of the absorbent article 10 as well as in a direction 501 that is substantially parallel to the central longitudinal axis 50 of the absorbent article 10 as well as in a z-direction that extends through the stack of layers in a direction that is substantially parallel to the garment-facing surface 2 and/or to the wearer-facing surface 4. The zones 630, 640 demarcate distinction regions of adhesive, including distinct regions of the outer cover adhesive 620 and distinct regions of the strength layer adhesive 650.

Referring to FIG. 17, these zones 630, 640 may comprise a number of distinct areas 632, 642. The areas 632, 642 may include one or more first joined areas 632 in the one or more outer zones 630 as well as at least one second joined area 642 in the at least one inner zone 640. Stated differently, the outer cover adhesive 620 may comprise an inner zone 640 positioned proximate to the central longitudinal axis 50 and an outer zone 630 positioned more distal from the central longitudinal axis 50 than the inner zone 640, wherein the outer zone 630 at least partially overlaps the side panel adhesive 610. These areas 632, 642 may extend in a direction 481 that is substantially parallel to the central lateral axis 48 of the absorbent article 10 as well as in a direction 501 that is substantially parallel to the central longitudinal axis 50 of the absorbent article 10. As illustrated in FIG. 17, these areas 632, 642 may extend across various portions of a wearer-facing side 43 of the outer cover material 40 and may encompass distinct portions of the outer cover adhesive 620. It is to be appreciated that similar areas may extend across various portions of a wearer-facing side of the nonwoven strength layer 700 and may encompass distinct portions of the strength layer adhesive 650. With respect to FIG. 17, the areas 632, 642 are shown extending over an attachment portion 410 of the outer cover material 40 that is attached to another layer, such as the backsheet 28 or the nonwoven strength layer 700. It is to be appreciated that the attachment portion 410 may extend over all or only a portion of the wearer-facing side 43 of the outer cover material 40. Similarly, an attachment portion of the nonwoven strength layer may extend over all or a portion of the strength layer. The outer cover adhesive 620 and/or the strength layer adhesive 650 may be deposited intermittently in the areas 632, 642. Additionally, the outer cover adhesive 620 and/or the strength layer adhesive 650 may be deposited differently in the first joined area 632 and the second joined area 642. In other words, these areas 632, 642 may comprise different “bond areas.”

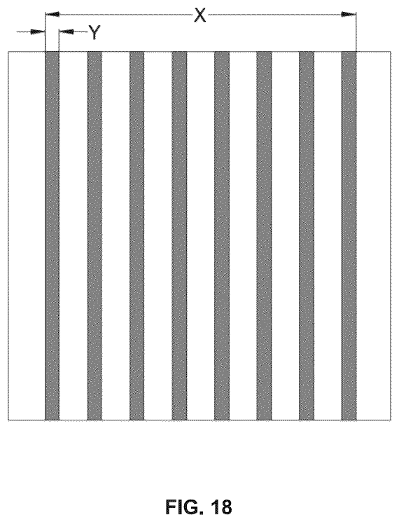

FIG. 18 is a schematic view of an adhesive pattern to illustrate a method of calculating a “bond area.” A “bond area” is defined as the fraction of a total “application area” covered by adhesive. As illustrated in FIG. 18, adhesive is applied in eight (8) strips, each extending across the entire length of an application area. If the total width of the application area is X and the width of each strip is Y, then the bond area is 8*Y/X.

Referring again to FIG. 17, the bond area of the first joined area 632 between the backsheet 28 and the outer cover material 40 in the outer zone 630 may be greater than the bond area of the second joined area 642 between the backsheet 28 and the outer cover material 40 in the inner zone 640. For example, the bond area of the first joined area 632 may be greater than the bond area of the second joined area 642 by about 1.3 times to about 5 times, or about 1.4 times to about 4.9 times, or about 1.5 times to about 4.8 times, or about 1.6 times to about 4.7 times, or about 1.7 times to about 4.6 times, or about 1.8 times to about 4.5 times, or about 1.9 times to about 4.4 times, or about 2 times to about 4.3 times, or about 2.1 times to about 4.2 times, or about 2.2 times to about 4.1 times, or about 2.3 times to about 4. times, or about 2.4 times to about 3.9 times, or about 2.5 times to about 3.8 times, or about 2.6 times to about 3.7 times, or about 2.7 times to about 3.6 times, or about 2.8 times to about 3.5 times, or about 2.9 times to about 3.4 times, or about 3. times to about 3.3 times, or about 3.1 times to about 3.2 times. Additionally or alternatively, the first joined area 632 may be at least 1.3 times greater than, or at least 1.6 times greater than, and less than 5 times greater than the second joined area 642.

### Cushion Material

One or more cushion materials may be provided in the absorbent articles 10 intermediate the topsheet and the backsheet. A cushion material may be a layer, laminate, or material that provides a cushiony feel when the absorbent article is touched from the garment-facing surface 2 or the wearer-facing surface 4. The cushion material may “mask” a grainy feel potentially caused by the absorbent material 72, such as superabsorbent polymers. The cushion material may also “mask” bodily exudates from being visible when viewing the wearer-facing surface 4 or the garment-facing surface 2 of the absorbent article 10. The cushion material may comprise nonwoven spunbond materials and may comprise one or more spunbond layers, such as two (SS), three (SSS), or four (SSSS), for example. The cushion material may optionally be hydroentangled. The cushion material may also not be hydroentangled, but still may comprise one more spunbond layers. The cushion material may have a basis weight in the range of about 15 gsm to about 50 gsm, about 20 gsm to about 45 gsm, about 25 gsm to about 40 gsm, about 30 to about 40 gsm, about 25 gsm, about 30 gsm, about 35 gsm, or about 40 gsm, according to the Basis Weight Test herein. The cushion material may be the layer forming the garment-facing side or the wearer-facing side of the core wrap/bag 74. The cushion material may also be placed within the core bag 74. The cushion material may be a separate material positioned intermediate the garment-facing side of the core bag 74 and the liquid impermeable backsheet 28. FIG. 19D is an example cross-sectional illustration of an absorbent article having an outer cover material and a cushion material. Referring to FIG. 19D, a cushion material 208 may be positioned intermediate a garment-facing surface of the core bag 74 and the backsheet 28. The cushion material may be free of a softening additive or may comprise a softening additive. The cushion materials, in combination with the outer cover materials discussed herein provides the absorbent articles with cushiony softness and loft. The cushion materials may have a thickness in the range of about 0.15 mm to about 0.5 mm, about 0.25 mm to about 0.30 mm, or about 0.35 mm to about 0.40 mm, or about 0.9 mm, specifically reciting all 0.1 mm increments within the specified ranges and all ranges formed therein or thereby. Hydroentangled cushion materials may be quite flexible compared to non-hydroentangled spunbond at the same basis weight. The cushion materials may have indicia printed, or otherwise applied, thereon. The cushion materials may comprise about 90% of 4 denier HDPE/PET bicomponent fibers and about 10% 3 denier PET. The cushion materials may be air through carded and may exhibit no bonding/embossing sites but only fiber-to-fiber bond intersections from the hot air bonding.

It should be understood that the various embodiments are not limited to the examples illustrated in the figures.

### Test Methods

### Side Panel Tensile Test

A suitable tensile tester interfaced with a computer such as MTS model Alliance RT/1 with TestWorks 4® software or equivalent is used. The tensile tester is located in a temperature-controlled room at 22° C.±2° C. and 50±10% relative humidity. The instrument is calibrated according to the manufacturer's instructions. The data acquisition rate is set to at least 50 Hertz. The grips used for the test are wider than the sample. Grips having 50.8 mm width may be used. The grips are air actuated grips designed to concentrate the entire gripping force along a single line perpendicular to the direction of testing stress having one flat surface and an opposing face from which protrudes a half round (radius=6 mm, e.g. part number: 56-163-827 from MTS Systems Corp.) or equivalent grips, to minimize slippage of the sample. The load cell is selected so that the forces measured are between 10% and 90% of the capacity of the load cell used. The initial distance between the lines of gripping force (gauge length) is set at 25.4 mm. The load reading on the instrument is zeroed to account for the mass of the fixture and grips.

A specimen measuring 50.8 mm (along the CD of the web or in the intended stretch direction of the laminate) by 25.4 mm (along the MD of the web or perpendicular to the intended stretch direction of the laminate) is cut either from the article component having the laminate, the component web (with the laminate therein) or the laminate web. The specimen is cut in a way that elasticized region is in the center and is at least 25.4 mm long in CD or in the intended stretch direction of the laminate. In the case when 50.8 mm long sample is not available, a specimen as small as 32 mm in CD or in the intended stretch direction can be used. The specimen is mounted into the grips in a manner such that there is no slack and the load measured is between 0.00 N and 0.02 N. The specimen is mounted in the center of the grips, such that the specimen direction of stretching is parallel to the applied tensile stress. The specimen is gripped in a way that elasticized region covers the entire gage length, i.e., elasticized region starts at the top grip and runs all the way to the bottom grip.

The specimen is extended at 508 mm/min, with a data acquisition rate of at least 50 Hertz, until the specimen breaks, typically >100% strain. The % strain is calculated from the length between grip lines L, and initial gauge length, L₀, using the following formula:

$$\% Strain = \frac{( L - {L}_{0} )}{{L}_{0}} \times 100$$

Each specimen is pulled until it ruptures (i.e. the post peak force response reaches a value less than 10% of the peak force). Break is defined as the point where the material fractures or ruptures, and force drops rapidly to zero value.

The testing is repeated for five separate specimens and the average and standard deviation of at least four specimens are reported. If, standard deviation recorded is higher than 5%, a new set of five specimens is run. The averaged values for each of the following are recorded: Elongation at 0.5N Load, the Elongation at 1.5N Load, the Elongation at 3.0N Load, the Load at Peak, and the Elongation at Peak. Elongation data are reported in % strain. Peak Force is normalized by the width of the sample and Load at Peak is reported as N/cm.

### Outer Cover Tensile Test

The Outer Cover Tensile Test is used to measure the tensile strength of the outer cover material along the lateral direction as present in an absorbent article. In this method, a constant-rate-of-extension tensile tester is used to pull specimens of sample outer cover material of interest to break, and the peak tensile load is recorded and reported. All measurements are done in an environment 23±2° C. and 50±5% RH on products and/or materials that had equilibrated to the laboratory environment for at least two hours prior to measurement.

Portions of outer cover material are removed from one or more like absorbent articles of interest. (The use of cryogenic freeze spray may be helpful in separating the outer cover material from the layer proximal to it.) Any region of the sheet to which other elements are additionally fastened (e.g. a landing zone) are cut away and excluded from further use. The axis on each sheet that is parallel to the lateral axis of the parent absorbent article is noted and is the axis of pull in the ensuing tensile tests. Enough like absorbent articles are used to yield five specimens of outer cover material, each 2.0″ in the dimension perpendicular to the axis of pull. (If there is difficulty removing outer cover material from finished articles and roll stock is available, it is acceptable to use roll stock, with the axis of pull being the cross direction of the roll.). Of the portions of outer cover material harvested, five specimens are cut. Specimens are at least 4.0″ long in the axis of pull and are 2.0″ perpendicular to the axis of pull.

A constant-rate-of-extension tensile tester is used to analyze each specimen. Each specimen is mounted in pneumatic grips with 1″×3″ grip faces of texture/material chosen to minimize chance of specimen slip. The gauge length is 3.0″ (76.2 mm), and the moving crosshead moves at 12.0″ (305 mm) per minute. The 2.0″ width of the sample is oriented perpendicular to the axis of crosshead travel, and the longer dimension (at least 4.0″) of the specimen is oriented along the axis of cross travel, with at least 0.5″ of material being held by each grip. Data are acquired and recorded at a frequency of at least 100 Hz. Each specimen is pulled to break, and the peak load during the entire pull is recorded in Newtons (N) as the peak load of that specimen.

The arithmetic mean of the peak load among all specimens is calculated and recorded to the nearest 0.01 N as the average peak load. The average peak load is then normalized by the specimen width of. 2.0″ (5.08 cm), and the result is reported in units of N/cm and to the nearest 0.01 N/cm as the outer cover material tensile strength.

### Hysteresis Test

The instrument is set up as described in the Side Panel Tensile Test Setup section above. Gage Length and Crosshead Speed are adjusted as per the test and table below. Data acquisition rate is set to at least 50 Hertz.

TABLE 1

| | Gage Length | Cross Head Speed |
|---|---|---|
| Elastic Definition test | 7 mm | 70 mm/min |
| Sample Hysteresis test | 25.4 mm | 254 mm/min |

### Sample Preparation:

For Elastic Definition Test: The specimen is cut with a dimension of 10 mm in the intended stretch direction of the laminate X 25.4 mm in the direction perpendicular to the intended stretch direction of the laminate. A specimen is collected from either an inelastic region or from an elastic region.

For Sample Hysteresis Test: The specimen is cut with a dimension of 50.8 mm (or minimum 32 mm) in the intended stretch direction of the laminate X 25.4 mm in the direction perpendicular to the intended stretch direction. A specimen is collected from an elastic region in such a way that it has corrugated area of at least 25.4 mm long in the stretch direction.

### Test Protocol

1. Pre-Load: Place the specimen in the grips such that the uniform width lies along a direction perpendicular to the gauge length direction. Secure the specimen in the upper grip, let the specimen hang slack, then close the lower grip. For sample hysteresis test, the corrugated length must cover the entire gage length (i.e., between the top grip and the bottom grip, there should not be an inelastic region or dead zone). Set the slack preload at 5 gram/force This means that the data collection starts when the slack is removed (at a constant crosshead speed of 13 mm/min) with a force of 5 gram force. Strain is calculated based on the adjusted gauge length (lini), which is the length of the specimen in between the grips of the tensile tester at a force of 5 gram-force. This adjusted gauge length is taken as the initial specimen length, and it corresponds to a strain of 0%. Percent strain at any point in the test is defined as the change in length relative to the adjusted gauge length, divided by the adjusted gauge length, multiplied by 100.
2. First cycle loading: Pull the specimen to the 100% strain at a constant cross head speed as described in Table 1. Record the load at the 75% strain, and at the 100% strain (Fini). Record the Length of specimen between the grips on first cycle at the 100% strain (lmax) to the nearest 0.001 mm.
3. First Hold: Hold the specimen at the 100% strain for 30 seconds. Record the load at the 100% strain (Fend) at the end of 30 seconds hold.
4. First cycle unloading: Return the crosshead to its starting position (initial sample length, lini) at a constant cross head speed as described in Table 1. Report the unload at the 50% strain.
5. Second Hold: Hold the specimen in the unstrained state for 1 minute.
6. Second cycle loading: Pull the specimen to the 100% strain at a constant cross head speed as described in Table 1. Record the Length of specimen between the grips at a second cycle load force of 7 gram-force (lext) to the nearest 0.001 mm
7. Second cycle hold and unload: Next, hold the specimen at the 100% strain for 30 seconds and then return the crosshead to its fresh starting position (before gage length adjustment) at a constant cross head speed as described in Table 1.

The testing is repeated for five separate specimens and the average and standard deviation of at least four specimens are reported. If, standard deviation recorded is higher than 5%, a new set of five specimens is run.

The measured and recorded forces, in Newtons (N), are the load forces at 75% and 100% strain in step (2), load force at 100% at the end of step (3), and the unload force at 50% strain in step (4). Recorded average lengths are lᵢₙᵢ, lₘₐₓ. and lₑₓₜ.
Force Relaxation %, which is defined as (Fᵢₙᵢ−F_{end})/Fᵢₙᵢ*100, is recorded to the nearest 0.01%.
% Set, which is defined as (lₑₓₜ−lᵢₙᵢ)/(lₘₐₓ−lᵢₙᵢ)*100%, is recorded to the nearest 0.01%.

$$Hysteresis Ratio = Load Force at 75 \% / Unload Force at 50 \%$$

The averaged values for each of the above is reported.

### Emtec Test

The Emtec Test is performed on garment-facing surfaces of outer cover nonwoven materials. In this test, TS7, TS750, and D values are measured using an Emtec Tissue Softness Analyzer (“Emtec TSA”) (Emtec Electronic GmbH, Leipzig, Germany) interfaced with a computer running Emtec TSA software (version 3.19 or equivalent). The Emtec TSA includes a rotor with vertical blades which rotate on the test sample at a defined and calibrated rotational speed (set by manufacturer) and contact force of 100 mN. Contact between the vertical blades and the test sample creates vibrations both in the blades and in the test piece, and the resulting sound is recorded by a microphone within the instrument. The recorded sound file is then analyzed by the Emtec TSA software to determine TS7 and TS750 values. The D value is a measure of sample stiffness and is based on the vertical distance required for the contact force of the blades on test sample to be increased from 100 mN to 600 mN. The sample preparation, instrument operation, and testing procedures are performed according to the instrument manufacturer's specifications.

### Sample Preparation

A test sample is prepared by cutting a square or circular portion of interest from the outer cover nonwoven material of an absorbent article. It is preferable that freeze spray is not used to remove the portion of the outer cover nonwoven material to be analyzed, though it is acceptable to use freeze spray in a distal region to aid in initiating the separation of layers. Test samples are cut to a length and width (diameter in the case of a circular sample) of no less than about 90 mm and no greater than about 120 mm to ensure the sample can be clamped into the TSA instrument properly. (If an absorbent article does not contain a sufficiently large area of the substrate of interest to extract a sample of the size specified above, it is acceptable to sample equivalent material from roll stock.) Test samples are selected to avoid unusually large creases or folds within the testing region. Six substantially similar replicate samples are prepared for testing.

All samples are equilibrated at TAPPI standard temperature and relative humidity conditions (23° C.±2° C.° and 50%±2%) for at least 2 hours prior to conducting the TSA testing, which is also conducted under TAPPI conditions.

### Testing Procedure

The instrument is calibrated according to the Emtec's instructions using the 1-point calibration method with the appropriate reference standards (so-called “ref2 samples,” or equivalent, available from Emtec).

A test sample is mounted in the instrument with the surface of interest facing upward, and the test is performed according to the manufacturer's instructions. The software displays values for TS7, TS750, and D when the automated instrument testing routine is complete. TS7 and TS750 are each recorded to the nearest 0.01 dB V² rms, and D is recorded to the nearest 0.01 mm/N. The test sample is then removed from the instrument and discarded. This testing procedure is performed individually on the corresponding garment-facing surfaces of interest of each of the six of the replicate samples.

The value of TS7, TS750, and D are each averaged (arithmetic mean) across the six sample replicates. The average values of TS7 and TS750 are reported to the nearest 0.01 dB V² rms. The average value of D is reported to the nearest 0.01 mm/N.

### Wrinkle Distribution Determination Method

The wrinkle distribution herein is determined by the following method. For each side panel to be tested, the number of wrinkles is counted over a length of 10 mm along the panel length (PL) with the laminate in relaxed state (i.e., without applying an extension force between ends thereof, preferably taken at its “unused” state wherein “unused” herein means without the panel ever having been previously stretched since manufacture). The total number of wrinkles is then divided by 10 in order to provide the number of wrinkles per unit length. A total of 4 random locations within the elastic region of the laminate may be measured accordingly and an average is calculated for each sample.

The 10 mm length is typically taken from a starting position that encompasses at least one full wrinkle (i.e., a complete wrinkle comprising a peak positioned between two consecutive troughs) and by doing so if at the opposite extremity of the 10 mm length only a partial wrinkle (i.e., not a complete wrinkle comprising a peak positioned between two consecutive troughs, e.g., the 10 mm end at a position corresponding to the peak of a wrinkle) is formed, this is not counted as a wrinkle within the present method. The above procedure is typically repeated for at least 4 samples of side panels and an average is calculated to provide the wrinkle distribution in wrinkles/mm as referred to herein.

As an example, the schematic of FIG. 19E illustrates wrinkles over a length of 10 mm along the panel length (PL) with the laminate in relaxed state. If the same is observed at the 4 locations within the same sample and after examining a total of at least 4 samples, the wrinkle distribution in this example is 2 wrinkles/mm. Of course, it is understood herein that in case of variations/differences in number of wrinkles observed at the respective locations and/or samples, the wrinkle distribution is taken as the average as explained above.

### Basis Weight Test

Each specimen is weighed to within ±0.1 milligram using a digital balance. Specimen length and width are measured using digital Vernier calipers or equivalent to within ±0.1 mm. All testing is conducted at 22±2° C. and 50±10% relative humidity. Basis weight is calculated using equation below.

$$Basis weight ( g / {m}^{2} ) = \frac{( Weight of specimen in grams )}{\begin{matrix}( Length of the specimen \\ in meter \times Width of the specimen in meter )\end{matrix}}$$

For calculating the basis weight of a substrate, a total 8 rectilinear specimens at least 10 mm×25 mm are used.

The average basis weight and standard deviation are recorded.

### Tearing Strength Test

The Tearing Strength Test is used to measure the force associated with forcibly separating or tearing a side panel from the chassis parallel to the lateral axis of the absorbent article. In this method, specimens that each contain a rear side panel, and the chassis immediate inboard of the side panel are excised and pulled to failure on a constant-rate-of-extension tensile tester. The peak tensile load is recorded and reported. All measurements are done in an environment 23±2° C. and 50±5% RH on products and/or materials that had equilibrated to the laboratory environment for at least two hours prior to measurement.

At least three like absorbent articles of interest are used to harvest specimens. Six like specimens are harvested. Contemplating an article flattened in lateral and longitudinal directions, specimens consistent of all layers in the thickness direction of the article, are bounded longitudinally by the inboard and outboard longitudinal extents of the rear side panels, and consist of all material laterally outboard of a position that is at least 1.0″ laterally inboard of the laterally inboard most position of each rear side panel. In other words, specimens isolate each side panel with a strip of chassis at least 1.0″ wide that can be gripped in the ensuing tensile pull. It may be noted that generally two symmetric specimens may be harvested from each absorbent article.

A constant-rate-of-extension tensile tester is used to pull each specimen to failure. The tensile tester is configured with the moving crosshead traveling at 50 mm/s and with pneumatic grips having 1″×6″ faces. (The 6″ width of the faces should be adjusted such that the width of the grips are at least as wide as the specimen as mounted.) Grip face texture/material is chosen so as to minimize slip. Data are acquired at a frequency of at least 100 Hz. Each specimen is mounted and interrogated as follows: The specimen is mounted to be pulled along the axis that was the lateral axis in the parent intact absorbent article. The chassis strip is mounted in the stationary lower grip, and what was the laterally outboardmost (as oriented in the parent absorbent article) portion of the side portion is clamped in the grip of the moving crosshead. The gauge length is not critical but is chosen such that the specimen can be mounted as described above and in a relaxed state. The tensile test is conducted and proceeds until the side panel separates from the chassis strip, and the peak load observed is recorded as the tearing strength of that specimen to the nearest 0.01 Newton (N).

All six specimens are interrogated in like fashion. The arithmetic mean of the resulting tearing strengths among all specimens is calculated and reported in N to the nearest 0.1 N as the tearing strength.

### EXAMPLES

The following examples are put forth to provide those of ordinary skill in the art with a complete disclosure and description of how to perform the methods, how to make, and how to use the compositions and compounds disclosed and claimed herein. Efforts have been made to ensure accuracy with respect to numbers (e.g., amounts, temperature, etc.), but some errors and deviations should be accounted for. The purpose of the following examples is not to limit the scope of the various embodiments, but merely to provide examples illustrating specific embodiments.

### Comparative Example 1

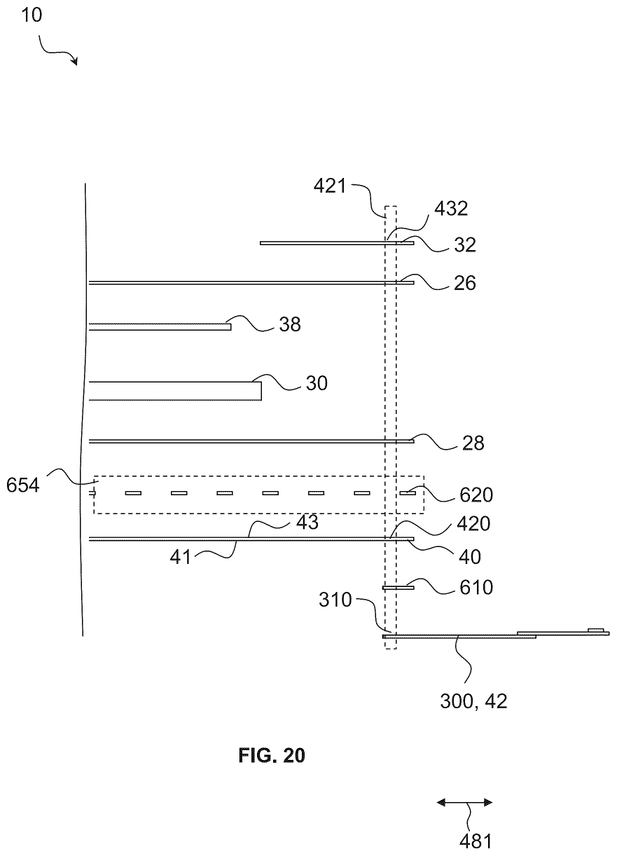

A purpose of this example is to illustrate and to test an absorbent article 10 having a structure as illustrated in FIG. 20. For simplicity, FIG. 20 shows half of the absorbent article 10. The other half of the absorbent article 10 was symmetrical to the half shown. The absorbent article 10 comprised barrier leg cuffs 32, a top sheet 26, acquisition materials 38, an absorbent core 30, a backsheet 28, an outer cover material 40, and a side panel 300. The barrier leg cuffs 32, the topsheet 26, the backsheet 28, the outer cover material 40, and the side panels 300 were mechanically bonded via a mechanical bond 421 as illustrated.

The barrier leg cuffs 32 were formed from a double-layered, spunbond nonwoven having a basis weight of about 13 gsm, determined according to the Basis Weight Test.

The top sheet 26 was formed from a double-layered, carded, and apertured nonwoven comprising two 25 gsm layers, determined according to the Basis Weight Test.

The acquisition materials 38 were formed from a combination of a carded wicking material having a basis weight of 43 gsm, determined according to the Basis Weight Test, and a cellulose fiber material having a basis weight of approximately 190 gsm, determined according to the Basis Weight Test.

The absorbent core 30 was made from super absorbent polymer (SAP) crystals encased in a nonwoven core bag adhered to layers of spunbond nonwovens.

The backsheet 28 was made from a fluid-impermeable film having a basis weight of 18 gsm, determined according to the Basis Weight Test.

The outer cover material 40 was formed from a thermally-bonded carded non-woven, having a basis weight of 27 gsm, determined according to the Basis Weight Test.

The outer cover material 40 had a tensile strength in a direction 481 substantially parallel to the central lateral axis 48 of 4.4 N/cm, measured according to the Outer Cover Tensile Test herein.

The side panel 300 was formed from a high-stretch elastomeric film ultrasonically bonded between two layers of spunbond nonwoven.

The absorbent article 10 had a total longitudinal length, L, taken in a direction 501 parallel to the central longitudinal axis 50 and the side panel 300 had a total longitudinal length, SPL, at its greatest length, taken in a direction 501 parallel to the central longitudinal axis 50. The SPL was 23% of L.

The outer cover material 40 was adhered to the backsheet 28 with an outer cover adhesive 620. The outer cover adhesive 620 was a hotmelt, pressure-sensitive adhesive, applied with a local basis weight of about 3 gsm.

The outer cover adhesive was distributed in a single pattern 1 mm wide slots uniformly spread across the material with 2 mm spacing between each slot having a bond area of 34%. It is to be appreciated that the with respect to patterns of adhesive the terms “slot” and “strip” may be used interchangeably.

The side panel 300 was adhered to a garment-facing side 41 of the outer cover material 40 via a side panel adhesive 610. The side panel adhesive 610 was a hotmelt, pressure-sensitive adhesive, applied with a local basis weight of about 10 gsm.

As determined according to the Tearing Strength Test herein, the side panel 300 exhibited a tearing strength of the attachment portion 310 of the side panel 300 from the outer cover material 40 as summarized in Table 2.

### Comparative Example 2

A purpose of this example is to illustrate and to test an absorbent article 10 having a structure as illustrated in FIG. 20. For simplicity, FIG. 20 shows half of the absorbent article 10. The other half of the absorbent article 10 was symmetrical to the half shown. The absorbent article 10 comprised barrier leg cuffs 32, a top sheet 26, acquisition materials 38, an absorbent core 30, a backsheet 28, an outer cover material 40, and a side panel 300. The barrier leg cuffs 32, the topsheet 26, the backsheet 28, the outer cover material 40, and the side panels 300 were mechanically bonded via a mechanical bond 421 as illustrated.

The barrier leg cuffs 32 were formed from a double-layered, spunbond nonwoven having a basis weight of about 13 gsm, determined according to the Basis Weight Test.

The top sheet 26 was formed from a double-layered, carded, and apertured nonwoven comprising two layers each having a basis weight of 25 gsm, determined according to the Basis Weight Test.

The acquisition materials 38 were formed from a combination of a carded wicking material having a basis weight of 43 gsm, determined according to the Basis Weight Test, and a cellulose fiber material having a basis weight of approximately 190 gsm, determined according to the Basis Weight Test.

The absorbent core 30 was made from super absorbent polymer (SAP) crystals encased in a nonwoven core bag adhered to layers of spunbond nonwovens].

The backsheet 28 was made from a fluid-impermeable film having a basis weight of 18 gsm, determined according to the Basis Weight Test.

The outer cover material 40 was formed from an air through-bonded carded non-woven, having a basis weight of 20 gsm, determined according to the Basis Weight Test.

The outer cover material 40 had a tensile strength in a direction 481 substantially parallel to the central lateral axis 48 of 1.6 N/cm, measured according to the Outer Cover Tensile Test herein.

The side panel 30 was formed from a high-stretch elastomeric film ultrasonically bonded between two layers of spunbond nonwoven. The side panel 300 was the same as in Comparative Example 1.

The absorbent article 10 had a total longitudinal length, L, taken in a direction 501 parallel to the central longitudinal axis 50 and the side panel 300 had a total longitudinal length, SPL, at its greatest length, taken in a direction 501 parallel to the central longitudinal axis 50. The SPL was 23% of L.

The outer cover material 40 was adhered to the backsheet 28 with an outer cover adhesive 620. The outer cover adhesive 620 was a hotmelt, pressure-sensitive adhesive, applied with a local basis weight of about 3 gsm.

The outer cover adhesive was distributed in a single pattern 1 mm wide slots uniformly spread across the material with 2 mm spacing between each slot having a bond area of 34%. It is to be appreciated that the with respect to patterns of adhesive the terms “slot” and “strip” may be used interchangeably.

The side panel 300 was adhered to a garment-facing side 41 of the outer cover material 40 via a side panel adhesive 610. The side panel adhesive 610 was a hotmelt, pressure-sensitive adhesive, applied with a local basis weight of about 10 gsm.

As determined according to the Tearing Strength Test herein, the side panel 300 exhibited a tearing strength of the attachment portion 310 of the side panel 300 from the outer cover material 40 as summarized in Table 2.

### Inventive Example 3

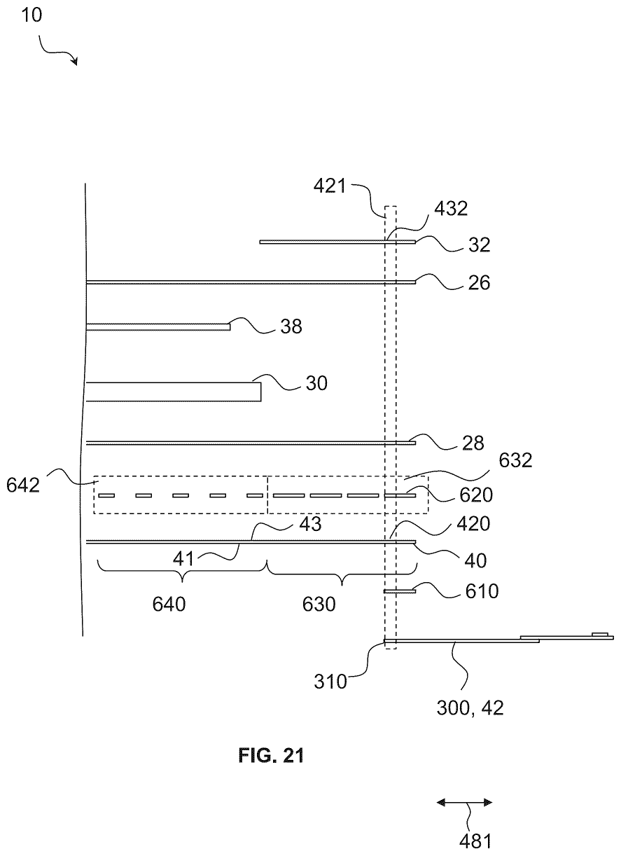

A purpose of this example is to illustrate and to test an absorbent article 10 having a structure as illustrated in FIG. 21. For simplicity, FIG. 21 shows half of the absorbent article 10. The other half of the absorbent article 10 was symmetrical to the half shown. The absorbent article 10 comprised barrier leg cuffs 32, a top sheet 26, acquisition materials 38, an absorbent core 30, a backsheet 28, an outer cover material 40, and a side panel 300. The barrier leg cuffs 32, the topsheet 26, the backsheet 28, the outer cover material 40, and the side panels 300 were mechanically bonded via a mechanical bond 421 as illustrated.

The barrier leg cuffs 32 were formed from a double-layered, spunbond nonwoven having a basis weight of about 20 gsm, determined according to the Basis Weight Test.

The top sheet 26 was formed from a double-layered, carded, and apertured nonwoven of 25 gsm each, determined according to the Basis Weight Test.

The acquisition materials 38 were formed from a combination of a carded wicking material having a basis weight of 43 gsm, determined according to the Basis Weight Test, and a cellulose fiber material having a basis weight of approximately 190 gsm, determined according to the Basis Weight Test.

The absorbent core 30 was made from super absorbent polymer (SAP) crystals encased in a nonwoven core bag adhered to layers of spunbond nonwovens].

The backsheet 28 was made from a fluid-impermeable film having a basis weight of 18 gsm, determined according to the Basis Weight Test.

The outer cover material 40 was formed from an air through-bonded carded non-woven, having a basis weight of 20 gsm, determined according to the Basis Weight Test.

The outer cover material 40 had a tensile strength in a direction 481 substantially parallel to the central lateral axis 48 of 1.7 N/cm, measured according to the Outer Cover Tensile Test herein.

The side panel 300 was formed from a high-stretch elastomeric film ultrasonically bonded between two layers of spunbond nonwoven. The side panel 300 was the same as for Comparative Examples 1 and 2.

The absorbent article 10 had a total longitudinal length, L, taken in a direction 501 parallel to the central longitudinal axis 50 and the side panel 300 had a total longitudinal length, SPL, at its greatest length, taken in a direction 501 parallel to the central longitudinal axis 50. The SPL was 23% of L.

The outer cover material 40 was adhered to the backsheet 28 with an outer cover adhesive 620. The outer cover adhesive 620 was a hotmelt, pressure-sensitive adhesive, applied with a local basis weight of about 3 gsm.

The outer cover adhesive 620 was distributed in three zones: two outer zones 630, and an inner zone 640. Each outer zone 630 comprised a first joined area 632, having a pattern with 2 mm wide slots uniformly spread across the material with 1 mm spacing between each slot and a bond area of 69%. The inner zone 640 comprised a second joined area 642, having a pattern with 1 mm wide slots uniformly spread across the material with 2 mm spacing between each slot and a bond area of 34%. It is to be appreciated that the with respect to patterns of adhesive the terms “slot” and “strip” may be used interchangeably.

The side panel 300 was adhered to a garment-facing side 41 of the outer cover material 40 via a side panel adhesive 610. The side panel adhesive 610 was a hotmelt, pressure-sensitive adhesive, applied with a local basis weight of about 10 gsm.

As determined according to the Tearing Strength Test herein, the side panel 300 exhibited a tearing strength of the attachment portion 310 of the side panel 300 from the outer cover material 40 as summarized in Table 2.

### Inventive Example 4

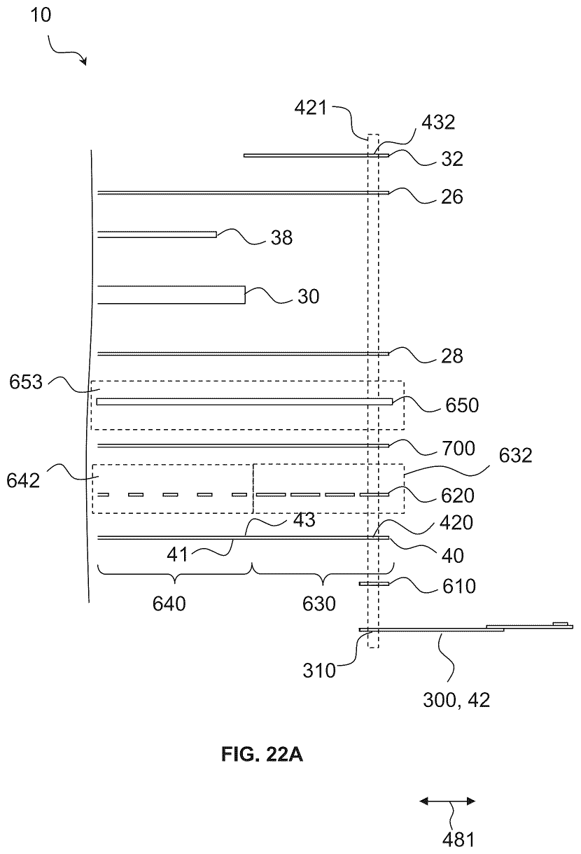

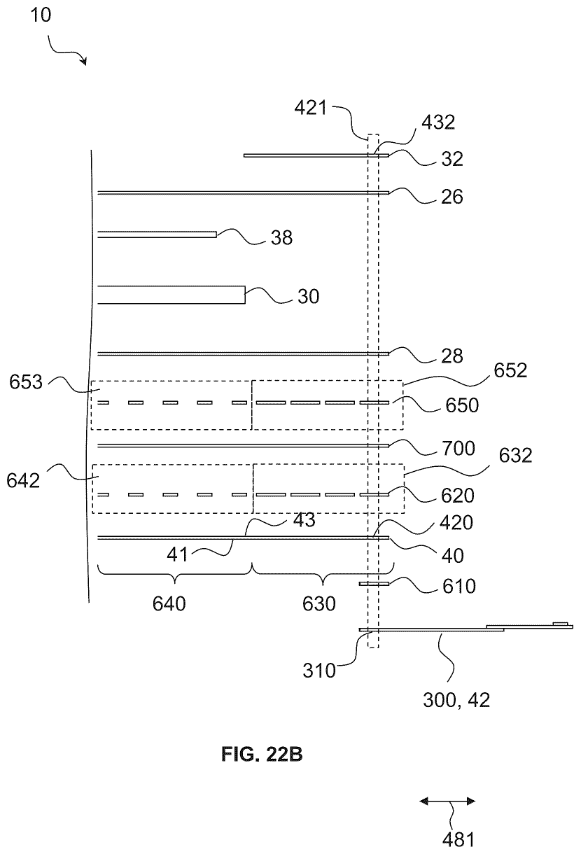

A purpose of this example is to illustrate and to test an absorbent article 10 having a structure as illustrated in FIG. 22A. For simplicity, FIG. 22A shows half of the absorbent article 10. The other half of the absorbent article 10 was symmetrical to the half shown. The absorbent article 10 comprised barrier leg cuffs 32, a top sheet 26, acquisition materials 38, an absorbent core 30, a backsheet 28, a nonwoven strength layer 700, an outer cover material 40, and a side panel 300. The barrier leg cuffs 32, the topsheet 26, the backsheet 28, the outer cover material 40, and the side panels 300 were mechanically bonded via a mechanical bond 421 as illustrated.

The barrier leg cuffs 32 were formed from a double-layered, spunbond nonwoven having a basis weight of about 20 gsm, determined according to the Basis Weight Test.

The top sheet 26 was formed from a double-layered, carded, and apertured nonwoven of 25 gsm each, determined according to the Basis Weight Test.

The acquisition materials 38 were formed from a combination of a carded wicking material having a basis weight of 43 gsm, determined according to the Basis Weight Test, and a cellulose fiber material having a basis weight of approximately 190 gsm, determined according to the Basis Weight Test.

The absorbent core 30 was made from super absorbent polymer (SAP) crystals encased in a nonwoven core bag adhered to layers of spunbond nonwovens.

The backsheet 28 was made from a fluid-impermeable film having a basis weight of 18 gsm, determined according to the Basis Weight Test.

The nonwoven strength layer 700 was a 17 gsm thermally-bonded spunbond nonwoven, determined according to the Basis Weight Test.

The outer cover material 40 was formed from an air through-bonded carded non-woven, having a basis weight of 20 gsm, determined according to the Basis Weight Test.

The outer cover material 40 had a tensile strength in a direction 481 substantially parallel to the central lateral axis 48 of 1.7 N/cm, measured according to the Outer Cover Tensile Test herein.

The side panel 300 was formed from a high-stretch elastomeric film ultrasonically bonded between two layers of spunbond nonwoven. The side panel 300 was the same as the side panels used in Comparative Examples 1, Comparative Example 2, Inventive Example 3, and Inventive Example 4.

The absorbent article 10 had a total longitudinal length, L, taken in a direction 501 parallel to the central longitudinal axis 50 and the side panel 300 had a total longitudinal length, SPL, at its greatest length, taken in a direction 501 parallel to the central longitudinal axis 50. The SPL was 23% of L.

The strength layer 700 was adhered to the backsheet 28 with a strength layer adhesive 650. The strength layer adhesive 650 was a hotmelt, pressure-sensitive adhesive.

The strength layer adhesive 650 was distributed in a single zone 653 and was applied as a uniform coating.

The outer cover material 40 was adhered to the strength layer 700 with an outer cover adhesive 620. The outer cover adhesive 620 was a hotmelt, pressure-sensitive adhesive, applied with a local basis weight of about 3 gsm.

The outer cover adhesive 620 was distributed in three zones: two outer zones 630, and an inner zone 640. Each outer zone 630 comprised a first joined area 632, having a pattern with 2 mm wide slots uniformly spread across the material with 1 mm spacing between each slot and a bond area of 69%. The inner zone 640 comprised a second joined area 642, having a pattern with 1 mm wide slots uniformly spread across the material with 2 mm spacing between each slot and a bond area of 34%. It is to be appreciated that the with respect to patterns of adhesive the terms “slot” and “strip” may be used interchangeably.

The side panel 300 was adhered to a garment-facing side 41 of the outer cover material 40 via a side panel adhesive 610. The side panel adhesive 610 was a hotmelt, pressure-sensitive adhesive, applied with a local basis weight of about 10 gsm.

As determined according to the Tearing Strength Test herein, the side panel 300 exhibited a tearing strength of the attachment portion 310 of the side panel 300 from the outer cover material 40 as shown in Table 2, below.

### Inventive Example 5

A purpose of this example is to illustrate an absorbent article 10 having a structure as illustrated in FIG. 22B. For simplicity, FIG. 22B shows half of the absorbent article 10. The other half of the absorbent article 10 may be symmetrical to the half shown. The absorbent article 10 may comprise barrier leg cuffs 32, a top sheet 26, acquisition materials 38, an absorbent core 30, a backsheet 28, a nonwoven strength layer 700, an outer cover material 40, and a side panel 300. The barrier leg cuffs 32, the topsheet 26, the backsheet 28, the outer cover material 40, and the side panels 300 may be mechanically bonded via a mechanical bond 421 as illustrated.

The barrier leg cuffs 32 may be formed from a double-layered, spunbond nonwoven having a basis weight of about 20 gsm, determined according to the Basis Weight Test.

The top sheet 26 may be formed from a double-layered, carded, and apertured nonwoven of 25 gsm each, determined according to the Basis Weight Test.

The acquisition materials 38 may be formed from a combination of a carded wicking material having a basis weight of 43 gsm, determined according to the Basis Weight Test, and a cellulose fiber material having a basis weight of approximately 190 gsm, determined according to the Basis Weight Test.

The absorbent core 30 may be made from super absorbent polymer (SAP) crystals encased in a nonwoven core bag adhered to layers of spunbond nonwovens.

The backsheet 28 may be made from a fluid-impermeable film having a basis weight of 18 gsm, determined according to the Basis Weight Test.

The nonwoven strength layer 700 may be a 17 gsm thermally-bonded spunbond nonwoven, determined according to the Basis Weight Test.

The outer cover material 40 may be formed from an air through-bonded carded non-woven, having a basis weight of 20 gsm, determined according to the Basis Weight Test.

The outer cover material 40 may have a tensile strength in a direction 481 substantially parallel to the central lateral axis 48 of 1.7 N/cm, measured according to the Outer Cover Tensile Test herein.

The side panel 300 may be formed from a high-stretch elastomeric film ultrasonically bonded between two layers of spunbond nonwoven. The side panel 300 may be the same as the side panels used in Comparative Example 1, Comparative Example 2, and Inventive Example 3.

The absorbent article 10 may have a total longitudinal length, L, taken in a direction 501 parallel to the central longitudinal axis 50 and the side panel 300 may have a total longitudinal length, SPL, at its greatest length, taken in a direction 501 parallel to the central longitudinal axis 50. The SPL was 23% of L.

The strength layer 700 may be adhered to the backsheet 28 with a strength layer adhesive 650. The strength layer adhesive 650 may be a hotmelt, pressure-sensitive adhesive.

The strength layer adhesive 650 may be distributed in three zones: two outer zones 630, and an inner zone 640. Each outer zone 630 may comprise a first joined area 652, having a pattern with 2 mm wide slots uniformly spread across the material with 1 mm spacing between each slot and a bond area of 69%. The inner zone 640 may comprise a second joined area 653, having a pattern with 1 mm wide slots uniformly spread across the material with 2 mm spacing between each slot and a bond area of 34%.

The outer cover material 40 may be adhered to the strength layer 700 with an outer cover adhesive 620. The outer cover adhesive 620 may be a hotmelt, pressure-sensitive adhesive, applied with a local basis weight of about 3 gsm.

The outer cover adhesive 620 may be distributed in three zones: two outer zones 630, and an inner zone 640. Each outer zone 630 may comprise a first joined area 632, having a pattern with 2 mm wide slots uniformly spread across the material with 1 mm spacing between each slot and a bond area of 69%. The inner zone 640 may comprise a second joined area 642, having a pattern with 1 mm wide slots uniformly spread across the material with 2 mm spacing between each slot and a bond area of 34%. It is to be appreciated that the with respect to patterns of adhesive the terms “slot” and “strip” may be used interchangeably.

The side panel 300 may be adhered to a garment-facing side 41 of the outer cover material 40 via a side panel adhesive 610. The side panel adhesive 610 maybe a hotmelt, pressure-sensitive adhesive, applied with a local basis weight of about 10 gsm.

### Results

Table 2 summarizes the side panel tearing force data for Examples 1-3. As can be seen from the results for Comparative Example 2, a soft material with low CD tensile strength usually results in a poor lateral ear strength. The invention, however, allows for a soft material with low CD tensile strength to exhibit excellent lateral ear strength.

TABLE 2

| Example | CD Tensile (N/cm) | Lateral Ear Strength (N) |
|---|---|---|
| Comparative 1 | 4.40 ± 0.33 | 61.8 ± 7.1 |
| Comparative 2 | 1.57 ± 0.15 | 40.3 ± 3.9 |
| Inventive 3 | 1.67 ± 0.12 | 60.4 ± 3.0 |

### Combinations

A1. A taped absorbent article comprising: a front waist region; a rear waist region; a crotch region intermediate the front waist region and the rear waist region; a central longitudinal axis; a central lateral axis; a garment-facing surface; a wearer-facing surface; a liquid permeable topsheet forming a portion of the wearer-facing surface; a liquid impermeable backsheet; an absorbent core positioned at least partially intermediate the topsheet and the backsheet, the absorbent core comprising an absorbent material; an outer cover material forming a portion of the garment-facing surface; wherein the outer cover material has a tensile strength in a direction substantially parallel to the central lateral axis in the range of about 0.5 N/cm to about 3 N/cm, according to the Outer Cover Tensile Test; a side panel that has a portion attached to a garment-facing side of the outer cover material in the rear waist region, wherein the side panel has an Elongation at 1.5N of about 25% or greater, according to the Side Panel Tensile Test; and wherein a tearing strength of the portion of the side panel from the outer cover material is at least 25 N, preferably at least 30N, preferably at least 35N, but less than 75N.
A2. The taped absorbent article of paragraph A1, wherein the taped absorbent article has a total longitudinal length, L, taken in a direction parallel to the central longitudinal axis, wherein the side panel has a total longitudinal length, SPL, at its greatest length, taken in a direction parallel to the central longitudinal axis, and wherein SPL is about 15% to about 30% of L, preferably about 19% to about 26% of L.
A3. The taped absorbent article of paragraph A1 or paragraph A2, wherein the outer cover material comprises a carded nonwoven material.
A4. The taped absorbent article of any of paragraphs A1-A3, wherein a denier of fibers of the outer cover material is in the range of about 0.4 to about 3.0 denier.
A5. The taped absorbent article of any of paragraphs A1-A4, wherein a basis weight of the outer cover material is in the range of about 15 gsm to about 40 gsm.
A6. The taped absorbent article of any of paragraphs A1-A5, comprising a mechanical bond at least partially overlapping a portion of the outer cover material and a portion of the side panel.
A7. The taped absorbent article of paragraph 6, comprising a leg cuff, wherein a portion of the leg cuff is overlapped by the mechanical bond.
A8. The taped absorbent article of any one of paragraphs A1-A7, wherein the side panel comprises a first nonwoven material, a second nonwoven material, and an elastic film positioned at least partially intermediate the first and second nonwoven materials.
A9. The taped absorbent article of paragraph A8, wherein the side panel comprises ultrasonic bonds joining the first nonwoven material to the second nonwoven material.
A10. The taped absorbent article of paragraph A9, wherein the elastic film is pre-activated prior to being joined to the first and second nonwoven materials.
A11. The taped absorbent article of any one of paragraphs A1-A10, wherein the side panel comprises a tape tab comprising fasteners.
A12. The taped absorbent article of any one of paragraphs A1-A11, wherein the side panel has an Unload Force at 50% of at least about 0.9 N and less than about 1.5 N, according to the Hysteresis Test.
A13. The taped absorbent article of any one of paragraphs A1-A12, wherein the side panel has a Hysteresis Ratio of at least about 2 and less than about 4, according to the Hysteresis Test.
A14. The taped absorbent article of any one of paragraphs A1-A13, comprising a side panel adhesive positioned intermediate a portion of the outer cover material and the portion of the side panel; an outer cover adhesive positioned between the outer cover material and the backsheet; the outer cover adhesive comprising an inner zone positioned proximate to the central longitudinal axis and an outer zone positioned more distal from the central longitudinal axis than the inner zone, wherein the outer zone at least partially overlaps the side panel adhesive; and wherein a first joined area between the backsheet and the outer cover material in the outer zone is at least 1.3 times greater than, preferably at least 1.6 times greater than, and less than 5 times greater than, a second joined area between the backsheet and the outer cover material in the inner zone.
A15. The taped absorbent article of any one of paragraphs A1-A14, wherein the garment-facing surface of the outer cover material has a TS7 value in the range of about 1 dB V2 rms to about 8 dB V2 rms.
B1. A taped absorbent article comprising: a front waist region; a rear waist region; a crotch region positioned intermediate the front waist region and the rear waist region; a central longitudinal axis; a central lateral axis; a garment-facing surface; a wearer-facing surface; a liquid permeable topsheet forming a portion of the wearer-facing surface; a liquid impermeable backsheet; an absorbent core positioned at least partially intermediate the topsheet and the backsheet, the absorbent core comprising an absorbent material; an outer cover material forming a portion of the garment-facing surface; wherein the outer cover material has a tensile strength in a direction substantially parallel to the central lateral axis in the range of about 0.5 N/cm to about 3 N/cm, according to the Outer Cover Tensile Test; wherein the outer cover material has a garment-facing side and a wearer-facing side; a side panel that has a portion attached to the garment-facing side of the outer cover material in the rear waist region, wherein the side panel has an Elongation at 1.5N of about 25% or greater, according to the Side Panel Tensile Test; a side panel adhesive positioned intermediate a portion of the outer cover material and the portion of the side panel; an outer cover adhesive positioned between the outer cover material and the backsheet; the outer cover adhesive comprising an inner zone positioned proximate to the central longitudinal axis and an outer zone positioned more distal from the central longitudinal axis than the inner zone, wherein the outer zone at least partially overlaps the side panel adhesive; and wherein a first joined area between the backsheet and the outer cover material in the outer zone is at least 1.3 times greater than, preferably at least 1.6 times greater than, and less than 5 times greater than, a second joined area between the backsheet and the outer cover material in the inner zone.
B2. The taped absorbent article of paragraph B1, wherein the outer cover material comprises a carded nonwoven material.
B3. The taped absorbent article of paragraph B1 or B2, wherein a denier of fibers of the outer cover material is in the range of about 0.4 to about 3.0 denier.
B4. The taped absorbent article of any of paragraphs B1-B3, wherein a basis weight of the outer cover material is in the range of about 15 gsm to about 40 gsm.
B5. The taped absorbent article of any one of paragraphs B1-B4, comprising a mechanical bond at least partially overlapping the outer cover adhesive in the outer zone, a portion of the outer cover material, the side panel adhesive, and the portion of the side panel.
B6. The taped absorbent article of paragraph B5, comprising a leg cuff, wherein a portion of the leg cuff is overlapped by the mechanical bond.
B7. The taped absorbent article of paragraph B5, comprising a nonwoven strength layer positioned intermediate the outer cover material and the backsheet.
B8. The taped absorbent article of paragraph B7, wherein the nonwoven strength layer is positioned intermediate the outer cover adhesive in the outer zone and a strength layer adhesive.
B9. The taped absorbent article of paragraph B7 or paragraph B8, wherein the mechanical bond at least partially overlaps the nonwoven strength layer and the strength layer adhesive.
B10. The taped absorbent article of any one of paragraphs B1-B9, wherein the side panel comprises a first nonwoven material, a second nonwoven material, and an elastic film positioned at least partially intermediate the first and second nonwoven materials.
B11. The taped absorbent article of paragraph B10, wherein the side panel comprises ultrasonic bonds joining the first nonwoven material to the second nonwoven material.
B12. The taped absorbent article of paragraph B11, wherein the elastic film is pre-activated prior to being joined to the first and second nonwoven materials.
B13. The taped absorbent article of any one of paragraphs B1-B12, wherein the outer cover adhesive is intermittent in a direction parallel to the central lateral axis.
B14. The taped absorbent article of any one of paragraphs B1-B13, wherein the side panel comprises a tape tab comprising a fastener.
B15. The taped absorbent article of any one of paragraphs B1-B14, wherein the side panel has an Unload Force at 50% of at least about 0.9 N and less than about 1.5 N, according to the Hysteresis Test.
B16. The taped absorbent article of any one of paragraphs B1-B15, wherein the side panel has a Hysteresis Ratio of at least about 2 and less than about 4, according to the Hysteresis Test.
B17. The taped absorbent article of any one of paragraphs B1-B16, wherein the taped absorbent article has a total longitudinal length, L, taken in a direction parallel to the central longitudinal axis, wherein the side panel has a total longitudinal length, SPL, at its greatest length, taken in a direction parallel to the central longitudinal axis, and wherein SPL is about 15% to about 30% of L, preferably about 19% to about 26% of L.
B18. The taped absorbent article of any paragraphs B1-B17, wherein the garment-facing surface of the outer cover material has a TS7 value in the range of about 1 dB V² rms to about 8 dB V² rms.
C1. A taped absorbent article comprising: a front waist region; a rear waist region; a crotch region intermediate the front waist region and the rear waist region; a central longitudinal axis; a central lateral axis; a garment-facing surface; a wearer-facing surface; a liquid permeable topsheet forming a portion of the wearer-facing surface; a liquid impermeable backsheet; an absorbent core positioned at least partially intermediate the topsheet and the backsheet, the absorbent core comprising an absorbent material; an outer cover material forming a portion of the garment-facing surface; wherein the outer cover material has a tensile strength in a direction substantially parallel to the central lateral axis in the range of about 0.5 N/cm to about 3 N/cm, according to the Outer Cover Tensile Test; wherein the outer cover material has a garment-facing side and a wearer-facing side; a side panel that has a portion attached to the garment-facing side of the outer cover material in the rear waist region, wherein the side panel has an Elongation at 1.5N of about 25% or greater, according to the Side Panel Tensile Test; a side panel adhesive positioned intermediate a portion of the outer cover material and the portion of the side panel; an outer cover adhesive positioned between the outer cover material and the backsheet; the outer cover adhesive comprising an inner zone positioned proximate the central longitudinal axis and an outer zone positioned more distal from the central longitudinal axis than the inner zone, wherein the outer zone at least partially overlaps the side panel adhesive; wherein a first joined area between the backsheet and the outer cover material in the outer zone is greater than a second joined area between the backsheet and the outer cover material in the inner zone; a nonwoven strength layer positioned intermediate the outer cover and the backsheet.
C2. The taped absorbent article of paragraph C1, wherein the side panel has an Unload Force at 50% of at least about 0.9 N and less than about 1.5 N, according to the Hysteresis Test.
C3. The taped absorbent article of paragraph C1 or paragraph C2, wherein the side panel has a Hysteresis Ratio of at least about 2 and less than about 4, according to the Hysteresis Test.
C4. The taped absorbent article of any of paragraphs C1-C3, wherein the taped absorbent article has total longitudinal length, L, taken in a direction parallel to the central longitudinal axis, wherein the side panel has a total longitudinal length, SPL, at its greatest length, taken in a direction parallel to the central longitudinal axis, and wherein SPL is about 15% to about 30% of L, preferably about 19% to about 26% of L.
C5. The taped absorbent article of any of paragraphs C1-C4, wherein the outer cover material comprises a carded nonwoven material.
C6. The taped absorbent article of any of paragraphs C1-C5, wherein a denier of fibers of the outer cover material is in the range of about 0.4 to about 3.0 denier.
C7. The taped absorbent article of any paragraphs C1-C6, wherein a basis weight of the outer cover material is in the range of about 15 gsm to about 40 gsm.
C8. The taped absorbent article of any paragraphs C1-C7, comprising a mechanical bond at least partially overlapping the outer cover adhesive in the outer zone, a portion of the outer cover material, the side panel adhesive, and the portion of the side panel.
C9. The taped absorbent article of paragraph C8, comprising a leg cuff, wherein a portion of the leg cuff is overlapped by the mechanical bond.
C10. The taped absorbent article of paragraph C8, wherein the mechanical bond at least partially overlaps the nonwoven strength layer and the strength layer adhesive.
C11. The taped absorbent article of any of paragraphs C1-C10, wherein the side panel comprises a first nonwoven material, a second nonwoven material, and an elastic film positioned at least partially intermediate the first and second nonwoven materials.
C12. The taped absorbent article of paragraph C11, wherein the side panel comprises ultrasonic bonds joining the first nonwoven material to the second nonwoven material.
C13. The taped absorbent article of paragraph C12, wherein the elastic film is pre-activated prior to being joined to the first and second nonwoven materials.
C14. The taped absorbent article of any of paragraphs C1-C13, wherein the outer cover adhesive is intermittent in a direction parallel to the central lateral axis.
C15. The taped absorbent article of any one of the preceding claims, wherein the side panel comprises a tape tab comprising fasteners.
C16. The taped absorbent article of any of paragraphs C1-C15, wherein the garment-facing surface of the outer cover material has a TS7 value in the range of about 1 dB V² rms to about 8 dB V² rms.

### Further Definitions and Cross-References

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. A taped absorbent article comprising:
a front waist region;
a rear waist region;
a crotch region intermediate the front waist region and the rear waist region;
a central longitudinal axis;
a central lateral axis;
a garment-facing surface;
a wearer-facing surface;
a liquid permeable topsheet forming a portion of the wearer-facing surface;
a liquid impermeable backsheet;
an absorbent core positioned at least partially intermediate the topsheet and the backsheet, the absorbent core comprising an absorbent material;
an outer cover material forming a portion of the garment-facing surface, wherein the outer cover material has a tensile strength in a direction substantially parallel to the central lateral axis in the range of about 0.5 N/cm to about 3 N/cm, according to the Outer Cover Tensile Test; and
a side panel that has a portion attached to a garment-facing side of the outer cover material in the rear waist region, wherein the side panel has an Elongation at 1.5N of about 25% or greater, according to the Side Panel Tensile Test;
wherein a tearing strength of the portion of the side panel from the outer cover material is at least 25 N, but less than 75N.

2. The taped absorbent article of claim 1, wherein the taped absorbent article comprises a taped absorbent article total longitudinal length taken in a direction parallel to the central longitudinal axis, wherein the side panel has a side panel total longitudinal length at its greatest length, taken in a direction parallel to the central longitudinal axis, and wherein the side panel total longitudinal length is about 15% to about 30% of the taped absorbent article total longitudinal length.

3. The taped absorbent article of claim 1, wherein the outer cover material comprises a carded nonwoven material.

4. The taped absorbent article of claim 1, wherein a denier of fibers of the outer cover material is in the range of about 0.4 to about 3.0 denier.

5. The taped absorbent article of claim 1, wherein a basis weight of the outer cover material is in the range of about 15 gsm to about 40 gsm.

6. The taped absorbent article of claim 1, comprising a mechanical bond at least partially overlapping a portion of the outer cover material and a portion of the side panel.

7. The taped absorbent article of claim 6, comprising a leg cuff, wherein a portion of the leg cuff is overlapped by the mechanical bond.

8. The taped absorbent article of claim 1, wherein the side panel comprises a first nonwoven material, a second nonwoven material, and an elastic film positioned at least partially intermediate the first and second nonwoven materials.

9. The taped absorbent article of claim 8, wherein the side panel comprises ultrasonic bonds joining the first nonwoven material to the second nonwoven material.

10. The taped absorbent article of claim 9, wherein the elastic film is pre-activated prior to being joined to the first and second nonwoven materials.

11. The taped absorbent article of claim 1, wherein the side panel comprises a tape tab comprising fasteners.

12. The taped absorbent article of claim 1, wherein the side panel has an Unload Force at 50% of at least about 0.9 N and less than about 1.5 N, according to the Hysteresis Test.

13. The taped absorbent article claim 1, wherein the side panel has a Hysteresis Ratio of at least about 2 and less than about 4, according to the Hysteresis Test.

14. The taped absorbent article of claim 1, comprising:
a side panel adhesive positioned intermediate a portion of the outer cover material and the portion of the side panel; and
an outer cover adhesive positioned between the outer cover material and the backsheet, the outer cover adhesive comprising:
an inner zone positioned proximate to the central longitudinal axis; and
an outer zone positioned more distal from the central longitudinal axis than the inner zone;
wherein the outer zone at least partially overlaps the side panel adhesive; and wherein a first joined area between the backsheet and the outer cover material in the outer zone is at least 1.3 times greater than, and less than 5 times greater than, a second joined area between the backsheet and the outer cover material in the inner zone.

15. The taped absorbent article of claim 1, wherein the garment-facing surface of the outer cover material has a TS7 value in the range of about 1 dB V2 rms to about 8 dB V2 rms.

16. A taped absorbent article comprising:
a front waist region;
a rear waist region;
a crotch region positioned intermediate the front waist region and the rear waist region;
a central longitudinal axis;
a central lateral axis;
a garment-facing surface;
a wearer-facing surface;
a liquid permeable topsheet forming a portion of the wearer-facing surface;
a liquid impermeable backsheet;
an absorbent core positioned at least partially intermediate the topsheet and the backsheet, the absorbent core comprising an absorbent material;
an outer cover material forming a portion of the garment-facing surface, wherein the outer cover material has a garment-facing side and a wearer-facing side;
a side panel that has a portion attached to the garment-facing side of the outer cover material in the rear waist region, wherein the side panel has an Elongation at 1.5N of about 25% or greater, according to the Side Panel Tensile Test;
a side panel adhesive positioned intermediate a portion of the outer cover material and the portion of the side panel; and
an outer cover adhesive positioned between the outer cover material and the backsheet, the outer cover adhesive comprising;
an inner zone positioned proximate to the central longitudinal axis; and
an outer zone positioned more distal from the central longitudinal axis than the inner zone;
wherein the outer zone at least partially overlaps the side panel adhesive;
wherein the outer cover material has a tensile strength in a direction substantially parallel to the central lateral axis in the range of about 0.5 N/cm to about 3 N/cm, according to the Outer Cover Tensile Test; and
wherein a first joined area between the backsheet and the outer cover material in the outer zone is at least 1.3 times greater than and less than 5 times greater than, a second joined area between the backsheet and the outer cover material in the inner zone.

17. The taped absorbent article of claim 16, wherein, comprising a mechanical bond at least partially overlapping the outer cover adhesive in the outer zone, a portion of the outer cover material, the side panel adhesive, and the portion of the side panel.

18. The taped absorbent article of claim 17, comprising a nonwoven strength layer positioned intermediate the outer cover material and the backsheet.

19. The taped absorbent article of claim 18, wherein the nonwoven strength layer is positioned intermediate the outer cover adhesive in the outer zone and a strength layer adhesive.

20. The taped absorbent article of claim 19, wherein the mechanical bond at least partially overlaps the nonwoven strength layer and the strength layer adhesive.
